(19) United States
(12) Patent Application Publication
(10) Pub. No.: US 2016/0101076 A1
(43) Pub. Date: Apr. 14, 2016

(54) Use of Sumoylation Inhibitors for Treating Cancer

(71) Applicant: Ronald J. WEIGEL, Iowa City, IA (US)

(72) Inventor: Ronald J. Weigel, Iowa City, IA (US)

(21) Appl. No.: 14/890,897

(22) PCT Filed: May 15, 2014
(86) PCT No.: PCT/US14/38193

§ 371 Date: Nov. 12, 2015

Related U.S. Application Data
(60) Provisional application No. 61/823,770, filed on May 15, 2013.

(51) Int. Cl. A61K 31/192; G01N 33/50; A61N 5/10; A61K 45/06

(52) U.S. Cl. CPC A61K 31/192; A61K 45/06; G01N 33/5011; A61N 5/10; A61N 2005/1098

(57) ABSTRACT

The present invention provides methods and reagents for treating cancer cells for therapeutic purposes, by contacting with a sumoylation inhibitor in a dose effective to block sumoylation of TFAP2A. In breast cancer cells the sumoylation inhibitor induces a basal to luminal shift in phenotype. Sumoylation inhibitors also reduce the number of cancer stem cells in a cancer cell population. Inhibition of sumoylation makes cancer cells more responsive to conventional chemotherapeutic therapy and radiation therapy and decreases recurrence or development of metastases.

### INTRODUCTION

The TFAP2C protein (TFAP2-gamma) is a sequence-specific DNA-binding transcription factor involved in the activation of several genes involved in mammary development, differentiation, and oncogenesis. It can act as either a homodimer or heterodimer with other family members and is induced during retinoic acid-mediated differentiation. It plays a role in the development of the eyes, face, body wall, limbs, and neural tube.

The sequence-specific DNA-binding protein interacts with inducible viral and cellular enhancer elements to regulate transcription of selected genes. AP-2 factors bind to the consensus sequence 5′-GCCNNNGGC-3′ and activate genes involved in a large spectrum of important biological functions. They also suppress a number of genes including MCAM/MUC18, C/EBP alpha and MYC.

Published studies have established a role for TFAP2C in the regulation of ESR1 (ERalpha) and ERBB2 (Her2) in breast carcinomas. TFAP2C has also been identified as a potential prognostic indicator for patients with breast tumors.

Sumoylation is a post-translational modification involved in various cellular processes, such as nuclear-cytosolic transport, transcriptional regulation, apoptosis, protein stability, response to stress, and progression through the cell cycle. The process of sumoylation involves the use of endogenous Small Ubiquitin-like Modifier (or SUMO) proteins, which are covalently attached to and detached from other proteins in cells to modify the function of those targeted proteins. SUMO proteins are similar to ubiquitin, and sumoylation is directed by an enzymatic cascade analogous to that involved in ubiquitination. In contrast to ubiquitin, SUMO is not used to tag proteins for degradation. Mature SUMO is produced when the last four amino acids of the C-terminus have been cleaved off to allow formation of an isopeptide bond between the C-terminal glycine residue of SUMO and an acceptor lysine on the target protein.

SUMO modification of proteins has many functions. Among the most frequent and best studied are protein stability, nuclear-cytosolic transport, and transcriptional regulation. Typically, only a small fraction of a given protein is sumoylated and this modification is rapidly reversed by the action of desumoylating enzymes. The SUMO-1 modification of RanGAP1 (the first identified SUMO substrate) leads to its trafficking from cytosol to nuclear pore complex. The SUMO modification of hNinein leads to its movement from the centrosome to the nucleus. In many cases, SUMO modification of transcriptional regulators correlates with inhibition of transcription.

There are 3 confirmed SUMO isoforms in humans; SUMO-1, SUMO-2, and SUMO-3. SUMO-2/3 show a high degree of similarity to each other and are distinct from SUMO-1. SUMO-4 shows similarity to -2/3 but it is as yet unclear whether it is a pseudogene or merely restricted in its expression pattern.

### PUBLICATIONS

Eloranta J J, Hurst H C. Transcription factor AP-2 interacts with the SUMO-conjugating enzyme UBC9 and is sumolated in vivo. J Biol Chem. 2002 Aug. 23; 277(34):30798-804.
Orso F, Corà D, Ubezio B, Provero P, Caselle M, Taverna D. Identification of functional TFAP2A and SP1 binding sites in new TFAP2A-modulated genes. BMC Genomics. 2010 Jun. 3; 11:355.
Zhou C, Li X, Du W, Feng Y, Kong X, Li Y, Xiao L, Zhang P. Antitumor effects of ginkgolic acid in human cancer cell occur via cell cycle arrest and decrease the Bcl-2/Bax ratio to induce apoptosis. Chemotherapy. 2010; 56(5):393-402.
Kessler J D, Kahle K T, Sun T, Meerbrey K L, Schlabach M R, Schmitt E M, Skinner S O, Xu Q, Li M Z, Hartman Z C, Rao M, Yu P, Dominguez-Vidana R, Liang A C, Solimini N L, Bernardi R J, Yu B, Hsu T, Golding I, Luo J, Osborne C K, Creighton C J, Hilsenbeck S G, Schiff R, Shaw C A, Elledge S J, Westbrook T F. A SUMOylation-dependent transcriptional subprogram is required for Myc-driven tumorigenesis. Science. 2012 Jan. 20; 335(6066):348-53.

### SUMMARY OF THE INVENTION

The present invention provides methods and reagents for treating cancer cells, including but not limited to carcinomas, for therapeutic purposes. In the methods of the invention, cancer cells, including without limitation cancer stem cells, are contacted with a dose of a sumoylation inhibitor effective to block sumoylation of TFAP2A. The inhibition of TFAP2A sumoylation alters the phenotype of the cancer cell, and also makes the cell more responsive to conventional cancer therapeutics.

In some embodiments, methods are provided for therapy of cancer with a sumoylation inhibitor as a single agent. In other embodiments methods are provided for a combination therapy for treating cancer with a sumoylation inhibitor and a chemotherapeutic agent, or a combination of a sumoylation inhibitor with radiation therapy. In some embodiments the sumoylation inhibitor is an anacardic acid or a derivative thereof, including, for example, ginkgolic acid. In such methods, an effective dose of a sumoylation inhibitor is administered to a cancer patient, in a combination with an effective dose of a chemotherapeutic agent or radiation therapy, wherein there is a decrease in the cancer cells present in the patient, for example a decrease in the cancer stem cells population. The synergy between treatment with sumoylation inhibitors and a chemotherapeutic agent or radiation therapy provide increased killing at equivalent or lower doses, and can sensitize otherwise resistant cells.

In some embodiments of the invention, breast carcinoma cells, for example a population of basal-type breast carcinoma cells, are contacted with a sumoylation inhibitor in a dose effective to induce a shift in the carcinoma phenotype from basal-type to luminal type breast carcinoma. The induced luminal-type carcinoma cells are more responsive to a chemotherapeutic agent or radiation therapy than the basal-type cells.

The methods may further comprise detecting a change in the phenotype of the cancer cells following administration of the sumoylation inhibitor, e.g. in breast cancer by detecting an increased percentage of cells expressing estrogen receptor a (ERα⁺ cells). Such methods may comprise monitoring one or more markers indicative the breast carcinoma phenotype, e.g. estrogen receptor, progesterone receptor, etc. Many carcinomas have a cancer stem cell population that are CD44⁺/CD24^{-/low}. A decrease in CD44 expression in the treated cell population, or a decrease in the number of CD44+ cancer stem cells can also be performed, where such a decrease is indicative of effective treatment with a sumoylation inhibitor.

The subject methods are used for prophylactic or therapeutic purposes. As used herein, the term “treating” is used to refer to treatment of pre-existing cancers, including those which are in apparent remission. The treatment of ongoing disease, in order to stabilize or improve the clinical symptoms of the patient, is of particular interest.

The sumoylation pathway is also used in screening assays to determine agents that are suitable for use in the methods of treatment, including without limitation screening derivatives and analogs of anacardic acids. Test compounds are screened for those that have the desired properties, through inhibition of sumoylation, which can involve inhibition of any of the molecular steps in the sumoylation pathway. Compounds of interest for screening include, without limitation, combinatorial libraries of small molecules; targeted modifications of compounds; environmental compounds, which can be derived from a wide variety of sources including plants, soil, water, foods; synthetic compounds such as chlorinated organics, polycyclic aromatic hydrocarbons, herbicides; pesticides; pharmaceuticals; and the like.

### BRIEF DESCRIPTION OF THE DRAWINGS

FIGS. 1A-1E. Functional Specificity of TFAP2C for the Luminal Cluster Genes. FIG. 1A. Primary ERα-positive breast cancer cells derived from patient samples were transduced with lentiviral vectors encoding shRNA specific for non-targeting (NT), TFAP2A (A) or TFAP2C (C). Knockdown of TFAP2A and TFAP2C was confirmed compared to NT (data for tumor 2 and 3 shown in FIG. 9A), *p<0.05 compared to NT. FIG. 1B. ERα RNA was assessed by RT-PCR; data for all three tumor isolates demonstrates that knockdown of TFAP2C specifically repressed ERα expression, * p<0.05 compared to NT. FIG. 1C. Western blot for ERα protein confirmed that ERα protein expression was repressed by knockdown of TFAP2C only. FIG. 1D. Functional effects on RNA expression of luminal genes, the basal genes, MMP14, CALD1 and CD44, and the TFAP2A-specific target gene CDKN1A/p21-CIP in MCF-7 cells after knockdown of either TFAP2A or TFAP2C; data demonstrates functional specificity of TFAP2C (additional luminal genes in FIG. 9B) with statistical differences shown comparing knockdown of TFAP2A vs. TFAP2C for all genes (p<0.05) and * p<0.05 compared to NT control. FIG. 1E. Western blot confirmed functional specificity for TFAP2C in regulation of luminal and basal genes.

FIGS. 2A-2F. ChIP of TFAP2A and TFAP2C with Functional Specificity of TFAP2C Mapped to Amino Terminus. FIG. 2A. ChIP-Seq demonstrates identical binding pattern comparing TFAP2A and TFAP2C to luminal target genes ESR1/ERα, FOXA1 and FREM2; red dot indicates peak analyzed in detail in part C. FIG. 2B. Western blot of MCF-7 cells transfected with empty vector (EV) or HA epitope tagged AP-2 constructs, TFAP2C (HA-C) or TFAP2A (HA-A), and probed with antibody shown. FIG. 2C. Real-time ChIP was performed with anti-HA antibody and precipitated chromatin amplified at off-target and on-target locations for ESR1/ERα, FOXA1 and FREM2 (Woodfield et al., 2010). Data confirm specific binding of TFAP2A and TFAP2C to peaks identified by ChIP-seq with minimal binding to off-peak sites. FIG. 2D. Schematic of TFAP2A (blue) and TFAP2C (yellow) showing homologous regions and chimeric AP-2 proteins generated (all chimeras generated using TFAP2C-mut, which is construct insensitive to the siRNA); AD: Activation Domain; Di: Dimerization Domain; DBD: DNA Binding Domain; assignment of functional domains described previously (Williams and Tjian, 1991a; Williams and Tjian, 1991b). FIG. 2E. Using endogenous ERα RNA expression as functional assay, MCF-7 cells were transfected with siRNA and expression vector as diagramed in D. The data show that rescue of ERα transcriptional activity maps to the amino half of the TFAP2C protein. FIG. 2F. Experiment identical to part E, except uses expression of endogenous FREM2 and maps functional effect to the first 128 amino acids of TFAP2C. *p<0.05 compared to normalized expression in untransfected control.

FIGS. 3A-3B. Yeast Two-hybrid Identifies Sumoylation Pathway Regulating Activity of TFAP2A on FREM2. FIG. 3A. Yeast two-hybrid screen using TFAP2A or TFAP2C as bait identified potential AP-2 interacting factors. Proteins from named genes are shown that were uniquely pulled out using either TFAP2A (blue) or TFAP2C (yellow) or were pulled out with both factors (green) as bait. FIG. 3B. Set of 21 factors was chosen for screening by knockdown with specific siRNAs in MCF-7 cells and assaying for effects on expression of endogenous FREM2 compared to NT siRNA (normalized to 1.0), *p<0.05 compared to NT. Two proteins identified as TFAP2A-interacting factors in yeast two-hybrid screen (PIAS1 and Ubc9/UBE2I) significantly induced FREM2 expression with knockdown.

FIGS. 4A-4I. Sumoylation Functionally Linked to AP-2 Activity. FIG. 4A. Ubc9 binds to TFAP2A and TFAP2C in GST pull-down. FIG. 4B. MCF-7 cells were transfected with expression vectors for Green Fluorescent Protein-AP-2 fusion proteins demonstrating nuclear expression of GFP fusion proteins with co-localization with DAPI nuclear stain. FIG. 4C. Co-IP of GFP-TFAP2A and GFP-TFAP2C confirms protein-protein interaction between Ubc9 and both AP-2 proteins. FIG. 4D. Expression of endogenous FREM2 RNA in MCF-7 cells transfected with siRNA (normalized to NT) indicated with western blots below. Data show that FREM2 expression is not responsive to TFAP2A; however, knockdown of Ubc9 induced FREM2 expression and induction is blocked with knockdown of TFAP2A showing that FREM2 expression responds to TFAP2A in absence of sumoylation pathway, p<0.05 compared to NT. FIG. 4E. Sumoylation using in vitro assay demonstrates wild type TFAP2A is sumoylated by SUMO-1, -2 or -3, whereas, TFAP2A K10R mutant has significantly reduced sumoylation. FIG. 4F. MCF-7 cells co-transfected with expression vector for TFAP2A or K10R mutant construct for SUMO-1, -2 or -3. Data show that wild-type TFAP2A is sumoylated in vivo, whereas the K10R mutant is not. FIG. 4G. Protein from MCF-7 cells was immunoprecipitated (IP) using IgG or anti-TFAP2A antibody; protein was eluted from beads (E) and prewashes (P1 and P2) were assayed; load is un-precipitated extract; western blot was probed with anti-SUMO2/3 antibody. FIG. 4H. TFAP2C was sumoylated in vitro with SUMO-1, -2 or -3; *indicates sumoylated form of TFAP2C. FIG. 4I. MCF-7 cells transfected with expression vectors for SUMO-1, -2 or -3 and western blot probed with TFAP2C shows evidence for sumoylation with SUMO-1; *indicates location of sumoylated TFAP2C.

FIGS. 5A-5E. Functional Effects of TFAP2A-K10R Mutant and Sumoylation Inhibition. FIG. 5A. Expression of luminal genes in MCF-7 cells transfected with wildtype TFAP2A or K10R mutant. K10R mutant induced luminal genes but wild-type TFAP2A did not. Both wild-type and K10R mutant TFAP2A induced CDKN1A/p21. FIG. 5B. Protein expression from western blots performed in triplicate with example of western blot below showing FREM2 protein expression induced by K10R but not wild-type TFAP2A. FIG. 5C. Expression of luminal cluster genes ESR1/ERα, KRT8 and FREM2 in sKD-C cells transfected with empty vector (EV) or expression vector for TFAP2A or K10R-TFAP2A mutant, with expression normalized to EV. K10R induced expression of luminal target genes, whereas, wild-type TFAP2A did not. FIG. 5D. Knockdown of Ubc9 or PIAS1 re-activated ERα and repressed CD44 expression in sKD-C cells. FIG. 5E. Treatment of sKD-C cells with ginkgolic acid (GA) re-activated FREM2 and ERα and repressed CD44 mRNA normalized to lowest value (top) and protein (bottom). For all panels, * indicates p<0.05 compared to normalized signal of 1.0. For CD44 expression in panel E, GA treated and untreated were also significantly different from each other, p<0.05.

FIGS. 6A-6C. Sumoylation Inhibitors Cleared Cancer Stem Cell Population. FIG. 6A. Treatment of s-KD-C, BT-549, BT-20 or cells derived from a primary basal cancer (Basal Cancer) with GA or anacardic acid (AA) inhibited CD44 expression by Western blot (top row) and significantly reduced the CD44⁺/CD24^{-/flow} population by FACS analysis (lower panels) but had no effect on the normal breast cell line MCF-10A. FIG. 6B/6C. Western blots showing that CD44 repression by GA and AA treatment was dependent upon expression of TFAP2A since knockdown of TFAP2A with siRNA abrogated effect of sumoylation inhibitors in sKD-C cells (FIG. 6B) and basal cell lines BT549 and BT20 (FIG. 6C).

FIGS. 7A-7D. Knockdown of Sumoylation Enzymes Repressed CD44 and Blocked SUMO Conjugation of TFAP2A. Knockdown of Ubc9 and PIAS1 by siRNA repressed expression of CD44 in BT549 (FIG. 7A) and BT20 (FIG. 7B) cells showing same effect as GA and AA. FIG. 7C/7D. Endogenous TFAP2A was examined by western blot in BT549 (basal) (FIG. 7C) and MCF-7 (luminal) (FIG. 7D) cells. The SUMO conjugated form of TFAP2A is seen in both cell types (denoted by *) and knockdown of either Ubc9 or PIAS1 significantly reduced SUMO-conjugated TFAP2A. MW shows molecular weight markers.

FIGS. 8A-8D. SUMO Inhibitors Repressed Outgrowth of Xenografts. FIG. 8A. Tumor free survival of nude mice (n=5 per group) inoculated with BT20 cells pretreated for 48 hours with either GA or AA compared to no pre-treatment; pretreated cells failed to form tumors. FIG. 8B. Tumor-free survival of nude mice (n=5 per group) inoculated with BT20 cells with animals gavaged with AA vs. vehicle. FIG. 8C. IOWA-1T cells were pre-treated with anacardic acid (AA) or vehicle and mice were followed until requiring euthanasia due to tumor size (Overall Survival). Pre-treatment with AA inhibited tumor formation, n=5 animals per group, p=0.002. FIG. 8D. Nude mice were inoculated with 1×10⁶, 5×10⁵, or 2.5×10⁵ IOWA-1T cells and animals were gavaged with either vehicle or AA; vehicle-gavaged mice developed tumors in 5, 10 and 12 days, respectively. Mice gavaged with AA failed to form tumors over the course of the experiment, n=3 animals per group, p=0.025.

FIG. 9A-9B. Effect of Knockdown of TFAP2C on Luminal Gene Expression. FIG. 9A. Additional data showing knockdown of TFAP2A and TFAP2C in cells from primary ERα-positive breast cancers, tumors 2 and 3, as indicated. FIG. 9B. Additional Luminal-associated genes, including the ERα-target gene, GREB1 (Ghosh et al., 2000), were repressed with knockdown of TFAP2C (C) but not TFAP2A (A). Knockdown of TFAP2A resulted in approximately 30% increase in GREB1 expression.

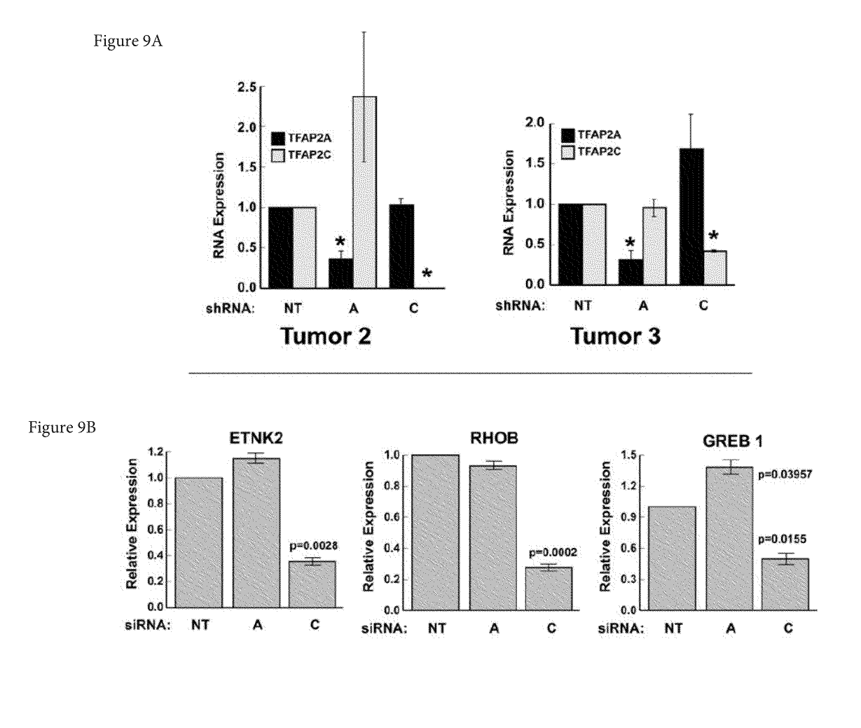

FIG. 10. FREM2 Expression in Breast Cancer Cell Lines and Primary Breast Cancers. FREM2 expression was characterized in a panel of cell lines by Western Blot (left) and as reported in Oncomine for FREM2 expression in primary breast cancers (right).

FIGS. 11A-11F. FREM2 is a Specific TFAP2C Target Gene. FIG. 11A. ChIP-Seq of FREM2 promoter demonstrates binding of TFAP2A and TFAP2C upstream of PolII loading site. FIG. 11B. Gel shift analysis localizes AP-2 binding site in FREM2 promoter. FIG. 11C. Knock down of TFAP2C (but not TFAP2A) repressed FREM2 expression. FIG. 11D. FREM2 expression is responsive to TFAP2C in SKBR-3 ER-negative cells. FIG. 11E. FREM2 expression is not estrogen responsive as noted by Tamoxifen treatment (TAM), whereas, RET is appropriately repressed with TAM. FIG. 11F. Cloned FREM2 promoter reporter demonstrates response to TFAP2C only.

FIG. 12. ChIP-Seq Data for Select Luminal Target Genes. ChIP-seq data for six luminal target genes ESR1, MUC1, FGFR4, KRT8, RET and MYB. For each gene, the first two rows are ChIP-seq data from our laboratory. The remaining data is from publically available datasets: TFAP2C-2: GSE23852; ESR/ERα: GSE45822; FOXA1: GSE23852; FOXM1: GSE40762; H3K4me1: GSE23701; H3K4me3: GSE23701; H3K27ac: GSE45822; H3K27me3: GSE23701. The data are in agreement that TFAP2A and TFAP2C bind to the same chromosomal locations. Furthermore, the location of AP-2 peaks agrees with other datasets for TFAP2C and co-localize with FOXA1 and ERα binding sites. Histone marks are consistent with active transcription and no significant binding in these regions was identified with H3K27me3, which is associated with chromatin silencing.

FIG. 13. Additional ChIP-Seq Data for AP-2 Target Genes. ChIP-seq data for additional AP-2 target genes including the luminal target genes FOXA1, GATA3, FREM2 and XBP1. ChIP-seq data for CD44 and CDKN1A are also shown. Format and dataset sources are identical as shown in FIG. 12.

FIG. 14A-14D. Size of Sumoylated TFAP2A and TFAP2C Proteins. Images of western blots shown in FIG. 4A-4I with protein ladders demonstrate the size of the sumoylated forms of AP-2 estimated to be approximately 70 kD±5 kD. For each gel, Magic Markers XP were used; * indicates the sumoylated form of AP-2 protein. FIG. 14A. First three lanes from the blot shown in FIG. 4E for TFAP2A sumoylated in vitro. FIG. 14B. MCF-7 extract followed by first lane of blot shown in FIG. 4F demonstrating size of sumoylated TFAP2A in vivo. FIG. 14C. Last two lanes of FIG. 4G showing sumoylated TFAP2A immunoprecipitated with anti-TFAP2A antibody and blotted with SUMO2/3 antibody. FIG. 14D. First two lanes of FIG. 4I showing sumoylated form of TFAP2C in vivo with SUMO-1.

FIG. 15A-15B. Sumoylated TFAP2A in Basal Cells Induced with Peroxide. Sumoylation of TFAP2A was analyzed in the basal cell line BT549 with and without treatment with peroxide. FIG. 15A. BT549 cells co-transfected with vectors for TFAP2A and either SUMO-1, -2 or -3 and treated without or with peroxide, as indicated. Induction of sumoylated TFAP2A was seen with peroxide treatment. Experiment in A was performed in the absence of the proteasome inhibitor MG-132. FIG. 15B. Similar experiments performed with MG-132 treatment either without or with peroxide. Peroxide increased the relative abundance of the base-line sumoylated form of TFAP2A and MG-132 enhanced identification of the sumoylated forms of TFAP2A.

FIG. 16. Anacardic Acid Inhibits SUMO-conjugated Proteins. Western blots were performed on protein extracts of BT549 cells treated with vehicle (V) or anacardic acid (AA) and reacted with antibodies for SUMO1, TFAP2A or GAPDH, as indicated. Equal amounts of protein were loaded in each lane. AA treatment significantly reduced the global presence of high molecular weight SUMO-conjugated proteins with similar amounts of free SUMO-1 noted. AA treatment specifically reduced the SUMO-conjugated form of TFAP2A (denoted by *). GAPDH control indicates equal loading of protein. MW indicates molecular weight markers.

FIGS. 17A-17D: qPCR, CD44 downregulation after treatment of colorectal CSC with anacardic acid, 48 h. FIG. 1B: qPCR, ALCAM (activated leucocyte cell adhesion molecule) after treatment of colorectal CSC with anacardic acid, 48 h. FIG. 17C: qPCR, EPCAM (epithelial cell adhesion molecule) after treatment of colorectal CSC with anacardic acid, 48 h. FIG. 17D: Western blot, CD44 downregulation after treatment of colorectal CSC with anacardic acid, 48 h.

FIG. 18: Western blot, CD44 downregulation, Panc-1 pancreatic carcinoma was treated with ginkgolic and anacardic acids, 96 h.

FIG. 19: Western blot, CD44 downregulation, 8505c thyroid carcinoma was treated with ginkgolic and anacardic acids, 96 h.

### DESCRIPTION OF THE SPECIFIC EMBODIMENTS

Cancer therapy is performed with sumoylation inhibitor compounds, optionally in combination with conventional cancer therapy. An effective dose of a sumoylation inhibitor is administered to a host suffering from a susceptible tumor, e.g. carcinomas, etc. Administration may be topical, localized or systemic, depending on the specific disease. The compounds are administered at a dosage that over a suitable period of time substantially reduces the tumor cell burden, while minimizing any side-effects.

Sumoylation Inhibitor.

The sumoylation pathway involves several steps including activation with E1 enzyme, conjugation with the E2 enzyme and ligation of the SUMO peptide with cooperative activity of the E2 and E3 ligases. Inhibitors of sumoylation can inhibit any of the steps of the sumoylation pathway.

In some embodiments a sumoylation inhibitor” or “SUMO inhibitor” refers to any small molecule inhibitor that binds one or more subunit of a sumoylation enzyme, thereby inhibiting the addition of a sumo protein to a target protein. Such small molecule inhibitors may also inhibit one or more sumoylation enzymes. Preferred sumoylation inhibitors have a high level of specificity to SUMO enzymes, thereby affecting sumoylation, but do not bind or have very low level or negligible binding to proteins found in the ubiquitination pathway.

In some embodiments the sumoylation inhibitor is an anacardic acid, or a derivative or analog thereof. Anacardic acid (C15:0) has the structure:

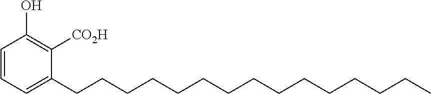 [SMILES: CCCCCCCCCCCCCCCc1cccc(O)c1C(=O)O]

Anacardic acids and derivatives thereof include, without limitation, Anacardic acid (C15:1); Anacardic acid (C15:2); Anacardic acid (C15:3); Anacardic acid (C24:1); Cardol (C12:0); N-isonicotinoyl-N′-8-[(2-carbohydroxy-3-hydroxy)phenyl]octanal hydrazine; N-isonicotinoyl-N′-8-[(2-carbohydroxy-3-hydroxy-6-nitro)phenyl]octanal hydrazine; N-(4-chloro-3-trifluoromethyl-phenyl)-2-ethoxy-6-pentadecyl-benzamide; N-(4-chloro-3-trifluoromethyl-phenyl)-2-ethoxy-benzamide; 2-isopropoxy-6-pentadecyl-N-pyridin-4-ylbenzamide; 2-ethoxy-N-(3-nitrophenyl)-6-pentadecylbenzamide; 2-ethoxy-6-pentadecyl-N-pyridin-4-ylbenzamide; 6-n-pentadecyl salicylic acid; 6-n-dodecylsalicylic acid; 6-n-heptadecylsalicylic acid; 5-[2-Ethoxy-5-(4-methylpiperazin-1-ylsulfonyl)-6-pentadecylphenyl]-1,6-dihydro-7H-pyrazolo[4, 3-d]pyrimidin-7-one; 5-[2-Methoxy-5-(4-methylpiperazin-1-ylsulfonyl)-6-pentadecylphenyl]-1,6-dihydro-7H-pyrazolo[4,3-d]pyrimidin-7-one; 2-hydroxy-6-[(8Z,11Z)-pentadeca-8,11,14-trienyl]benzoic acid.

A derivative of particular interest is gingkolic acid, (Z)-6-(Pentadec-8-enyl)-2-hydroxybenzoic acid.

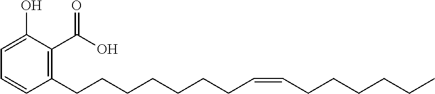 [SMILES: CCCCCC/C=C\CCCCCCCc1cccc(O)c1C(=O)O]

Analogues of interest include those of Formula I:

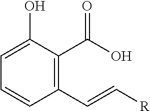

Where R is an aliphatic or aromatic moiety, for example CH₂(CH₂)ₙCH₃; where n is from 1-10; benzyl; etc.

Examples of sumoylation inhibitors are described, for example in US 20130245032 A1, Hemshekhar et al. (2012) Basic and Clinical Pharmacology & Toxocology 110(2):122-132; Ghizzoni et al. (2010) Bioorg. Med. Chem 18(16):5826-34; Kusio-Kobialka et al. (2013) Anticancer Agents Med Chem 13(5):762-7; Zhang et al. (2011 Molecules 16: 4059-4069; each herein specifically incorporated by reference.

An effective dose of a sumoylation inhibitor is a dose that reduces the concentration of SUMO-conjugated form of TFAP2A in a cancer cell, e.g. reduces by about 10%, by about 20%, by about 30%, by about 40%, by about 50%, by about 60%, by about 70%, by about 80%, by about 80%, by about 90%, or more. The effective dose can also be monitored by the effect of down-regulating sumoylation of TFAP2A in a population of cancer cells, where in a population of cell the expression of CD44 is down-regulated, such that the number of CD44⁺ cells in the population is reduced by about 10%, by about 20%, by about 30%, by about 40%, by about 50%, by about 60%, by about 70%, by about 80%, by about 80%, by about 90%, or more. Where the population of cancer cells is a population of basal-type breast carcinoma cells, the effective dose may be monitored by the phenotypic change from basal-type to luminal type, for example as exemplified by expression of ERα, wherein in a population of basal-type breast carcinoma cells, treatment with an effective dose of a sumoylation inhibitor increased the number of ERα⁺ cells at least about 2-fold, at least about 3-fold, at least about 5-fold, at least about 10-fold, or more.

Alternatively, an effective dose of a sumoylation inhibitor can be measured as the dose that results in killing of cancer cells. In some embodiments, the dose of sumoylation inhibitor as a single agent is effective to kill at least about 25% of the cancer cells present in a population, more usually at least about 50% killing, and may be about 90% or greater of the cells present in a population.

In other embodiments the effective dose of a sumoylation inhibitor can be measured as the dose that results in altering the phenotype of a cancer cell to be susceptible to chemotherapy or radiation therapy, where the number of cells in a population susceptible to a conventional dose of chemotherapy or radiation is increased at least about 2-fold, at least about 3-fold, at least about 5-fold, at least about 10-fold, or more, relative to an untreated population.

In some embodiments, a population of cells is monitored for one or more of the above activities, i.e. alteration of phenotype, decrease of CD44⁺ cells, increase in susceptibility to chemotherapy or radiation, etc.

The susceptibility of a particular tumor cell population to killing with the combined therapy may be determined by in vitro testing, as detailed in the experimental section. Typically a culture of the tumor cell is combined with a combination of a chemotherapeutic or radiation therapy and a sumoylation inhibitor at varying concentrations for a period of time sufficient to allow the active agents to work, usually between about one hour and one week. For in vitro testing, cultured cells from a biopsy sample of the tumor may be used. The viable cells left after treatment are then counted.

In some embodiments of the invention, a combination therapy is provided. The combined used of sumoylation inhibitors and chemotherapeutics agent has the advantages that the required dosages for the individual drugs is lower, and the effect of the different drugs complementary. Depending on the patient and condition being treated and on the administration route, the sumoylation inhibitors may be administered in dosages of 0.001 mg to 5 mg/kg body weight per day. The range is broad, since in general the efficacy of a therapeutic effect for different mammals varies widely with doses typically being 20, 30 or even 40 times smaller (per unit body weight) in man than in the rat. Similarly the mode of administration can have a large effect on dosage. Thus for example oral dosages in the rat may be ten times the injection dose. The dosage for the chemotherapeutic agent will be equal to, or less than the conventional dosage for that agent.

A typical dosage may be a solution suitable for intravenous administration; a tablet taken from two to six times daily, or one time-release capsule or tablet taken once a day and containing a proportionally higher content of active ingredient, etc. The time-release effect may be obtained by capsule materials that dissolve at different pH values, by capsules that release slowly by osmotic pressure, or by any other known means of controlled release.

Those of skill will readily appreciate that dose levels can vary as a function of the specific compound, the severity of the symptoms and the susceptibility of the subject to side effects. Some of the specific compounds are more potent than others. Preferred dosages for a given compound are readily determinable by those of skill in the art by a variety of means. A preferred means is to measure the physiological potency of a given compound.

For use in the subject methods, sumoylation inhibitors may be formulated with other pharmaceutically active agents, particularly other anti-metastatic, anti-tumor or anti-angiogenic agents. Angiostatic compounds of interest include angiostatin, endostatin, carboxy terminal peptides of collagen alpha (XV), etc. Cytotoxic and cytostatic agents of interest include adriamycin, alkeran, Ara-C, BICNU, busulfan, CNNU, cisplatinum, cytoxan, daunorubicin, DTIC, 5-FU, hydrea, ifosfamide, methotrexate, mithramycin, mitomycin, mitoxantrone, nitrogen mustard, velban, vincristine, vinblastine, VP-16, carboplatinum, fludarabine, gemcitabine, idarubicin, irinotecan, leustatin, navelbine, taxol, taxotere, topotecan, etc.

It is contemplated that the composition will be obtained and used under the guidance of a physician for in vivo use. To provide a synergistic effect in a combined therapy, a chemotherapeutic agent, or radiation therapy, and the sumoylation inhibitor can be delivered together or separately, and simultaneously or at different times within the day.

The dose will vary depending on the specific cytotoxic agent utilized, type of tumor, patient status, etc., at a dose sufficient to substantially ablate the tumor cell population, while maintaining patient viability. Treatment will generally be continued until there is a substantial reduction, e.g. at least about 50%, decrease in the tumor burden, and may be continued until there are essentially no tumor cells detected in the body.

Tumors of interest for treatment include carcinomas, e.g. breast, colon, colorectal, prostate, pancreatic, melanoma, ductal, endometrial, stomach, dysplastic oral mucosa, invasive oral cancer, non-small cell lung carcinoma, thyroid, transitional and squamous cell urinary carcinoma, etc.; neurological malignancies, e.g. neuroblastoma, gliomas, etc.; hematological malignancies, e.g. childhood acute leukemia, non-Hodgkin's lymphomas, chronic lymphocytic leukemia, malignant cutaneous T-cells, mycosis fungoides, non-MF cutaneous T-cell lymphoma, lymphomatoid papulosis, T-cell rich cutaneous lymphoid hyperplasia, bullous pemphigoid, discoid lupus erythematosus, lichen planus, etc.; and the like.

Breast cancer is of particular interest. The majority of breast cancers are adenocarcinoma subtypes. Ductal carcinoma in situ is the most common type of noninvasive breast cancer. In DCIS, the malignant cells have not metastasized through the walls of the ducts into the fatty tissue of the breast. Infiltrating (or invasive) ductal carcinoma (IDC) has metastasized through the wall of the duct and invaded the fatty tissue of the breast. Infiltrating (or invasive) lobular carcinoma (ILC) is similar to IDC, in that it has the potential metastasize elsewhere in the body. About 10% to 15% of invasive breast cancers are invasive lobular carcinomas. Of interest in the treatment of breast cancer are the various types of breast cancer, e.g. luminal A; luminal B; HER2-type; and basal-type, triple negative carcinomas. Luminal A cancers are ER⁺ and/or PR⁺, HER2⁻, low Ki67e. Luminal B cancers are ER⁺ and/or PR⁺, HER2⁺ (or HER2⁻ with high Ki67). Basal-type cancers are triple negative, i.e. ER⁻, PR⁻, HER2⁻ and are typically resistant to conventional chemotherapy or radiation. Her2 type are ER⁻, PR⁻, HER2+.

The host, or patient, may be from any mammalian species, e.g. primate sp., particularly humans; rodents, including mice, rats and hamsters; rabbits; equines, bovines, canines, felines; etc. Animal models are of interest for experimental investigations, providing a model for treatment of human disease.

Pharmaceutical Formulations:

sumoylation inhibitors can be incorporated into a variety of formulations for therapeutic administration. More particularly, the compounds of the present invention can be formulated into pharmaceutical compositions by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intracheal, etc., administration. The active agent may be systemic after administration or may be localized by the use of regional administration, intramural administration, or use of an implant that acts to retain the active dose at the site of implantation.

In pharmaceutical dosage forms, the compounds may be administered in the form of their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The compounds can be utilized in aerosol formulation to be administered via inhalation. The compounds of the present invention can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more compounds of the present invention. Similarly, unit dosage forms for injection or intravenous administration may comprise the compound of the present invention in a composition as a solution in sterile water, normal saline or another pharmaceutically acceptable carrier.

Implants for sustained release formulations are well-known in the art. Implants are formulated as microspheres, slabs, etc. with biodegradable or non-biodegradable polymers. For example, polymers of lactic acid and/or glycolic acid form an erodible polymer that is well-tolerated by the host. The implant containing sensitizer is placed in proximity to the site of the tumor, so that the local concentration of active agent is increased relative to the rest of the body.

The term “unit dosage form”, as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Drug screening methods are used to identify agents that inhibit sumoylation for use in the methods of the invention. Of particular interest are screening assays for agents that have a low toxicity for human cells. A wide variety of assays may be used for this purpose, including labeled in vitro protein-protein binding assays, electrophoretic mobility shift assays, immunoassays for protein binding, and the like. For example, the effect of an agent on tumor cell phenotype with respect to susceptibility to conventional chemotherapeutic agents, expression of CD44, expression of estrogen receptor in breast carcinoma cells, expression of reporters based on response genes, etc. can be measured in a screening assay. A reduction on the sumo conjugated form of TFAP2A in a cancer cell can be measured. The susceptibility of a particular tumor cell population to killing with the combined therapy may be determined by in vitro testing, as detailed in the experimental section. Typically a culture of the tumor cell is combined with a combination of a chemotherapeutic or radiation therapy and a sumoylation inhibitor at varying concentrations for a period of time sufficient to allow the active agents to work, usually between about one hour and one week. For in vitro testing, cultured cells from a biopsy sample of the tumor may be used. The viable cells left after treatment are then counted.

The purified protein may also be used for determination of three-dimensional crystal structure, which can be used for modeling intermolecular interactions. The sumoylation pathway involves several steps including activation with E1 enzyme, conjugation with the E2 enzyme and ligation of the SUMO peptide with cooperative activity of the E2 and E3 ligases. Inhibitors of sumoylation can involve screening to inhibit any of the steps of the sumoylation pathway.

The term “agent”, for example a candidate agent tested in a screening assay, as used herein describes any molecule, e.g. protein or pharmaceutical, with the capability of inhibiting sumoylation, particularly inhibiting sumoylation of TFAP2A in a cancer cell. Generally a plurality of assay mixtures are run in parallel with different agent concentrations to obtain a differential response to the various concentrations. Typically, one of these concentrations serves as a negative control, i.e. at zero concentration or below the level of detection.

Candidate agents encompass numerous chemical classes, though typically they are organic molecules, preferably small organic compounds having a molecular weight of more than 50 and less than about 2,500 daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In some embodiments the candidate agent is a derivative or analog of anacardic acid.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Where the screening assay is a binding assay, one or more of the molecules may be joined to a label, where the label can directly or indirectly provide a detectable signal. Various labels include radioisotopes, fluorescers, chemiluminescers, enzymes, specific binding molecules, particles, e.g. magnetic particles, and the like. Specific binding molecules include pairs, such as biotin and streptavidin, digoxin and antidigoxin etc. For the specific binding members, the complementary member would normally be labeled with a molecule that provides for detection, in accordance with known procedures.

A variety of other reagents may be included in the screening assay. These include reagents like salts, neutral proteins, e.g. albumin, detergents, etc. that are used to facilitate optimal protein-protein binding and/or reduce non-specific or background interactions. Reagents that improve the efficiency of the assay, such as protease inhibitors, nuclease inhibitors, anti-microbial agents, etc. may be used. The mixture of components are added in any order that provides for the requisite binding. Incubations are performed at any suitable temperature, typically between 4 and 40° C. Incubation periods are selected for optimum activity, but may also be optimized to facilitate rapid high-throughput screening. Typically between 0.1 and 1 hours will be sufficient.

The compounds having the desired pharmacological activity may be administered in a physiologically acceptable carrier to a host for treatment of cancer, etc., or to otherwise enhance function. The agents may be administered in a variety of ways, orally, topically, parenterally e.g. subcutaneously, intraperitoneally, by viral infection, intravascularly, etc. Topical treatments are of particular interest. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

The agents can be used in native form or can be modified to form a chemical derivative. As used herein, a molecule is said to be a “chemical derivative” of another molecule when it contains additional chemical moieties not normally a part of the molecule. Such moieties may improve the molecule's solubility, absorption, biological half life, etc. The moieties may alternatively decrease the toxicity of the molecule, eliminate or attenuate any undesirable side effect of the molecule, etc. Moieties capable of present invention can be administered concurrently with, prior to, or following the administration of the other agent.

The agents are administered to the mammal in a pharmaceutically acceptable form and in a therapeutically effective concentration. A composition is said to be “pharmacologically acceptable” if its administration can be tolerated by a recipient patient. Such an agent is said to be administered in a “therapeutically effective amount” if the amount administered is physiologically significant. An agent is physiologically significant if its presence results in a detectable change in the physiology of a recipient patient.

The agents of the present invention can be formulated according to known methods to prepare pharmaceutically useful compositions, whereby these materials, or their functional derivatives, are combined in admixture with a pharmaceutically acceptable carrier vehicle. Suitable vehicles and their formulation, inclusive of other human proteins, e.g., human serum albumin, are described, for example, in Remington's Pharmaceutical Sciences (16th ed., Osol, A., Ed., Mack, Easton Pa. (1980)). In order to form a pharmaceutically acceptable composition suitable for effective administration, such compositions will contain an effective amount of one or more of the agents of the present invention, together with a suitable amount of carrier vehicle.

Additional pharmaceutical methods may be employed to control the duration of action. Control release preparations may be achieved through the use of polymers to complex or absorb one or more of the agents of the present invention. The controlled delivery may be exercised by selecting appropriate macromolecules (for example polyesters, polyamino acids, polyvinyl, pyrrolidone, ethylenevinylacetate, methylcellulose, carboxymethylcellulose, or protamine, sulfate) and the concentration of macromolecules as well as the methods of incorporation in order to control release. Another possible method to control the duration of action by controlled release preparations is to incorporate agents of the present invention into particles of a polymeric material such as polyesters, polyamino acids, hydrogels, poly(lactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatine microcapsules; and poly(methylmethacylate) microcapsules, respectively, or in colloidal drug delivery systems, for example, liposomes, albumin microspheres, microemulsions, nanoparticles, and nanocapsules or in macroemulsions.

The following examples are offered by way of illustration and not by way of limitation.

### EXPERIMENTAL

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

The TFAP2C/AP-2γ transcription factor is required to maintain the luminal mammary phenotype. Functional specificity of TFAP2C was mapped to the activation domain and a non-selective screen identified the requirement of the sumoylation pathway to maintain TFAP2C-specific gene regulation. Disruption of the sumoylation pathway by knockdown of sumoylation enzymes, mutation of the SUMO-target lysine of TFAP2A, or treatment with sumoylation inhibitors induced a basal to luminal transition. Sumoylation inhibitors cleared the cancer stem cell population characterizing basal cancers but had no effect on normal mammary epithelial cells. These findings establish a critical role for sumoylation in regulating the transcriptional mechanisms that maintain the basal cancer phenotype. Sumoylation inhibitors offer a novel therapeutic approach for the treatment of aggressive basal breast cancer.

Clinical breast cancer subtypes are characterized by patterns of gene expression that predict outcome and response to therapy. Luminal breast cancers express steroid hormone receptors and tend to be hormone responsive. By comparison, basal breast cancers are not hormone responsive, display an expansion of cancer stem cells and have a worse prognosis. Herein, we show that TFAP2C has a unique role compared to other AP-2 family members in maintaining patterns of gene expression that characterize luminal cancers. We report the novel finding that the sumoylation pathway is required to maintain TFAP2C specific gene regulation of the luminal expression cluster. Sumoylation inhibitors induce a transition from the basal towards a luminal breast cancer phenotype thus forming the basis for new treatment strategies.

Breast cancer has an incidence of 226,000 and accounts for approximately 40,000 deaths annually in the US. There has been an improvement in survival for women with breast cancer, though patients with locally advanced or metastatic disease continue to have a poor prognosis. The clinical subtypes of breast cancer are defined by the expression of estrogen receptor-alpha (ERα), progesterone receptor (PgR) and amplification and overexpression of c-ErbB2/HER2. The four common molecular subtypes of breast cancers include the Luminal A (ERα/PgR+, HER2−), Luminal B (ERα/PgR+, HER2+), HER2 (ER</PgR−, Her2+) and triple-negative (ERα/PgR−, HER2−). The luminal breast cancer subtypes (comprising approximately 75% of breast cancer in postmenopausal women) are characterized by the expression of a set of ERα-associated genes (Sorlie et al., 2001).

The triple-negative breast cancer subtype is a heterogeneous group that represents 10-20% of breast cancers (Bertucci et al., 2012; Lehmann et al., 2011). The triple-negative subtypes have an aggressive clinical course and do not respond to therapy effective for cancers that express ERα or HER2. Hence, there has been intense research focus on understanding the molecular characterization of this group with the goal of defining novel molecular targets (Bertucci et al., 2012). Detailed molecular profiling has allowed further subclassification of the triple-negative breast cancer phenotypes into at least six distinct subtypes including basal-like 1, basal-like 2, immunomodulatory, mesenchymal-like, mesenchymal stem-like and luminal androgen receptor subtypes (Lehmann et al., 2011). Other proposed sub-classifications of the triple negative breast cancer phenotype have identified a claudin-low subgroup characterized by the relatively reduced expression of genes involved in cell adhesion and formation of tight junctions (Herschkowitz et al., 2007; Valentin et al., 2012).

Basal-like breast cancers are further distinguished from luminal cancers by frequent mutations of TP53, gene expression patterns characteristic of epithelial-to-mesenchymal transition (EMT) and an increase in the percentage of cancer stem cells (CSC) (Bertucci et al., 2012; Valentin et al., 2012). TFAP2C (AP-2γ) is a member of the developmentally regulated family of AP-2 factors that include five members—TFAP2A (AP-2α), TFAP2B (AP-2β), TFAP2C (AP-2γ), TFAP2D (AP-2δ) and TFAP2E (AP-2ε) (Bosher et al., 1996; Feng and Williams, 2003; Moser et al., 1995; Williams et al., 1988; Zhao et al., 2001). TFAP2C binds to a GC-rich consensus sequence in the promoters of target genes through a helix-loop-helix motif in the DNA binding domain (Eckert et al., 2005). Analysis of a ChIP-seq data set for TFAP2C defined the consensus site as the nine base sequence SCCTSRGGS (S=G/C, r=NG) (Woodfield et al., 2010), which closely matches the previously defined optimal in vitro binding 6 site (McPherson and Weigel, 1999).

AP-2 factors are expressed early in differentiation of the ectoderm and specify cell fates within the epidermis and neural crest (Hoffman et al., 2007; Li and Cornell, 2007). Within the adult mammary gland, TFAP2C is expressed in the luminal and myoepithelial cells (Cyr et al., 2014; Friedrichs et al., 2005; Friedrichs et al., 2007). Overexpression of TFAP2A or TFAP2C in mouse mammary epithelial cells (MMEC) results in lactation failure with hypoplasia of the alveolar mammary epithelium during pregnancy (Jager et al., 2003; Zhang et al., 2003). Conditional knockout of the mouse homolog of TFAP2C, Tcfap2c, in MMEC promoted aberrant growth of the mammary tree leading to a reduction in the luminal cell population and concomitant gain of the basal cell population at maturity (Cyr et al., 2014).

In tumor models, both TFAP2A and TFAP2C are important to cell proliferation, establishment of colonies in soft agar, cell migration and xenograft outgrowth (Orso et al., 2008). In breast cancer, AP-2 factors regulate expression of both ERα and Her2. TFAP2C regulates expression of ERα as well as other ERα-associated genes characteristic of luminal breast cancer (Cyr et al., 2014; deConinck et al., 1995; McPherson et al., 1997; Woodfield et al., 2007). TFAP2A and TFAP2C induce expression of the cloned HER2/ErbB2 promoter (Begon et al., 2005; Bosher et al., 1996; Delacroix et al., 2005; Yang et al., 2006). TFAP2C bound to the HER2 promoter and knockdown of TFAP2C reduced HER2 expression (Ailan et al., 2009). In BT474 breast carcinoma cells, TFAP2A and TFAP2C coordinately regulate HER2 expression (Allouche et al., 2008) and a correlation has been established between AP-2 expression and the expression of HER2 in primary breast cancers (Allouche et al., 2008; Pellikainen et al., 2004; Turner et al., 1998).

There is 83% similarity between TFAP2A and TFAP2C with 76% identity in the carboxyl-half of the proteins containing the DNA binding and dimerization domains (McPherson et al., 1997). In neural crest development, TFAP2A and TFAP2C appear to have complementary and overlapping roles (Hoffman et al., 2007). However, in breast cancer models, TFAP2C was found to have a unique role in regulation of ESR1/ERα gene expression, which was functionally distinct from the effects of TFAP2A (Woodfield et al., 2007). Furthermore, recent findings have highlighted a critical role for TFAP2C in maintaining the luminal phenotype through the induction of luminal-associated genes and repression of basal-associated genes (Cyr et al., 2014). It remains to be seen if TFAP2A has a similar effect on the expression of luminal genes. Furthermore, if the role of TFAP2C were functionally distinct, it would be of critical importance to understand the molecular basis for transcriptional specificity of luminal gene regulation. We expect that mechanisms regulating patterns of gene expression in breast cancer would provide important insight into novel strategies for drug development. With these considerations in mind, we sought to confirm the functional differences between TFAP2A and TFAP2C in regulation of luminal gene expression and to determine the molecular basis for functional specificity of TFAP2C-mediated gene regulation.

### Results

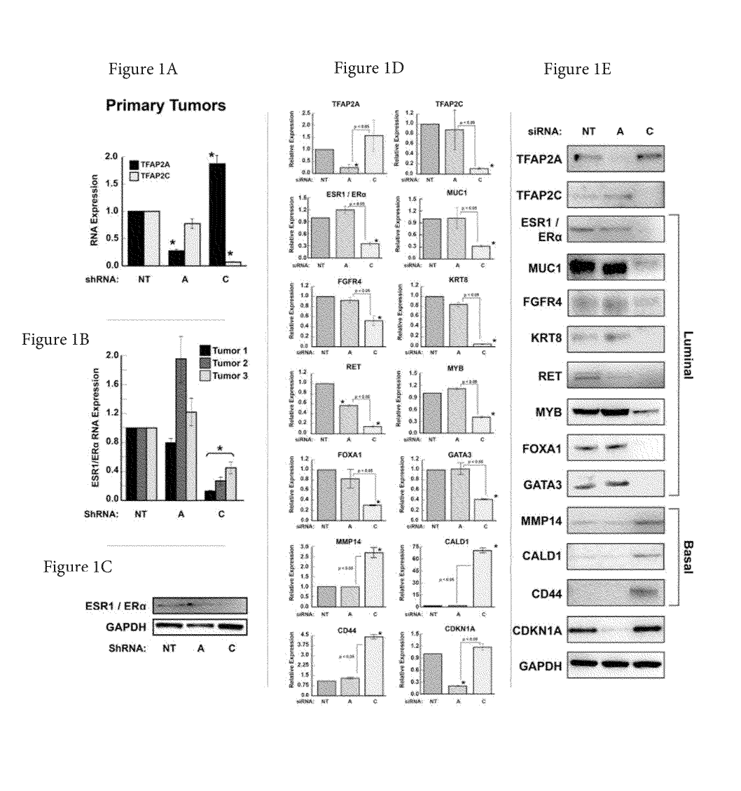

Functional Specificity of TFAP2C for the Luminal Gene Expression Cluster. In order to validate the unique functional role of TFAP2C in primary human cancer, we obtained fresh tumor tissue from patients with ERα-positive breast cancer. Tumor-derived breast cancer cells were transduced with lentiviruses encoding TFAP2A, TFAP2C or non-targeting shRNA. As seen in FIGS. 1A-C and 9, knockdown of TFAP2C but not TFAP2A repressed expression of ERα, confirming that TFAP2C has unique functional effects with regard to ESR1/ERα gene regulation and that the cell line models are reflective of gene regulation in primary human breast cancer. Using MCF-7 cells, a more expansive examination of luminal gene targets was performed. The luminal breast cancer subtype expresses a set of luminal-associated genes including ESR1/ERα, MUC1, FGFR4, KRT8, RET, MYB, FOXA1 and GATA-3 (Kao et al., 2009). Knockdown of TFAP2C repressed expression of luminal genes, as noted by analysis of RNA (FIG. 1D, 9B) and protein (FIG. 1E), whereas, knockdown of TFAP2A had minimal or no effect. The basal target genes, MMP14, CALD1 and CD44, which are overexpressed in basal cancers, were repressed by TFAP2C but not TFAP2A. By contrast, a known TFAP2A target gene, CDKN1A/p21-CIP (Scibetta et al., 2010; Woodfield et al., 2007), was responsive to TFAP2A only (FIG. 1D, E). Hence, although TFAP2A has the ability to induce certain genes, it lacks functional activity with regard to the luminal-associated gene expression cluster.

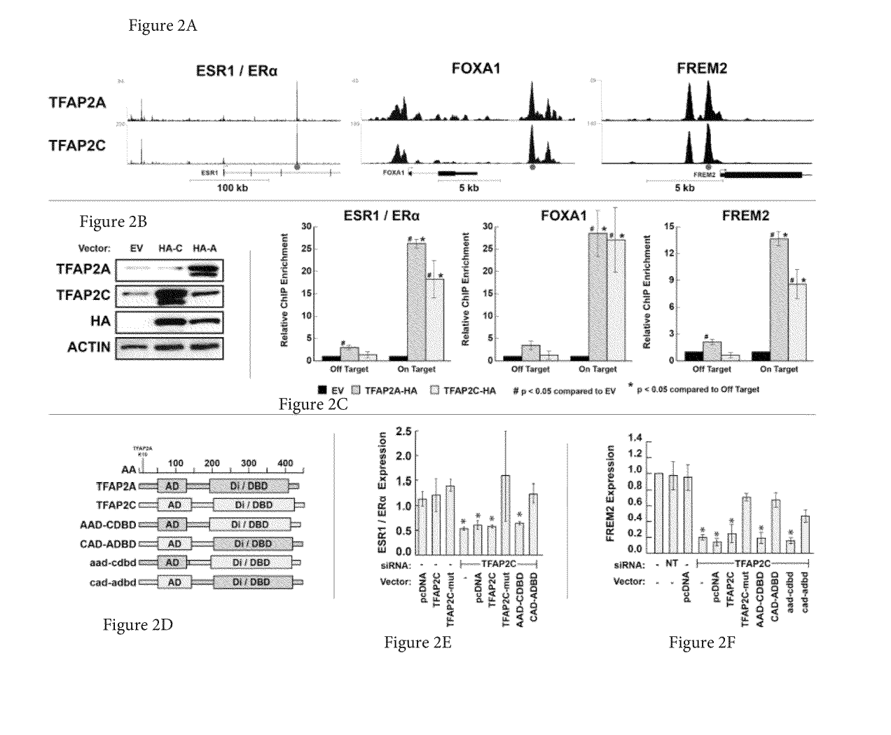

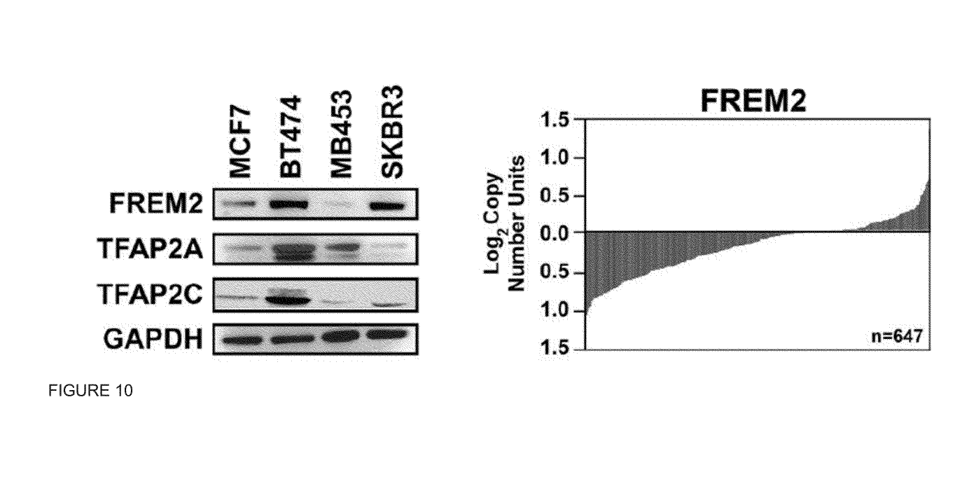

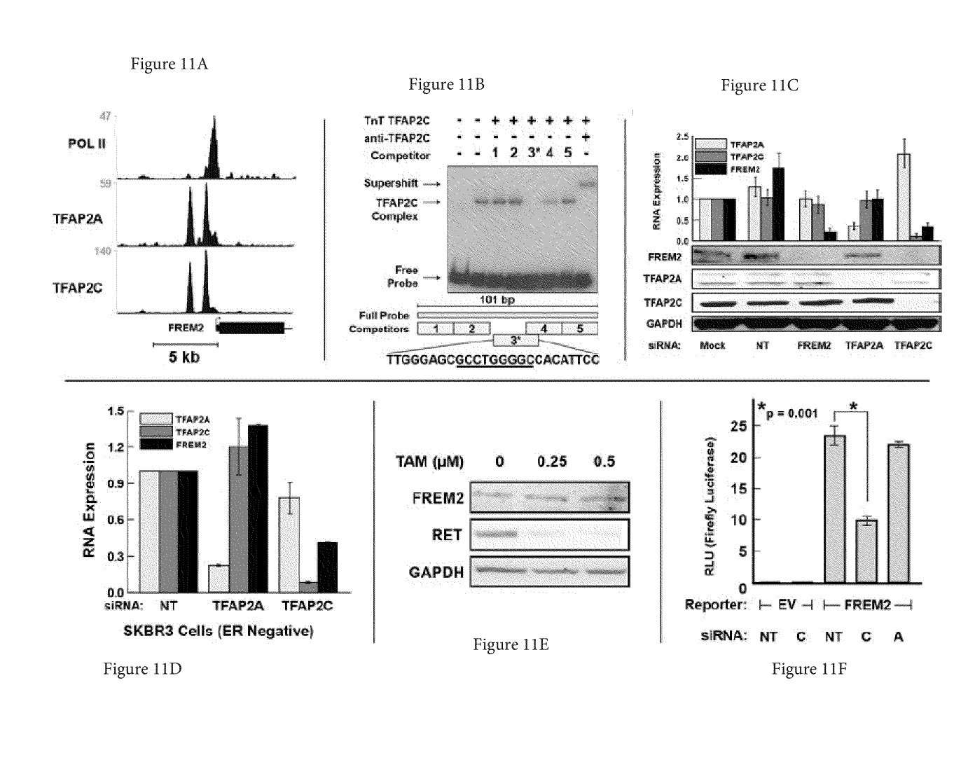

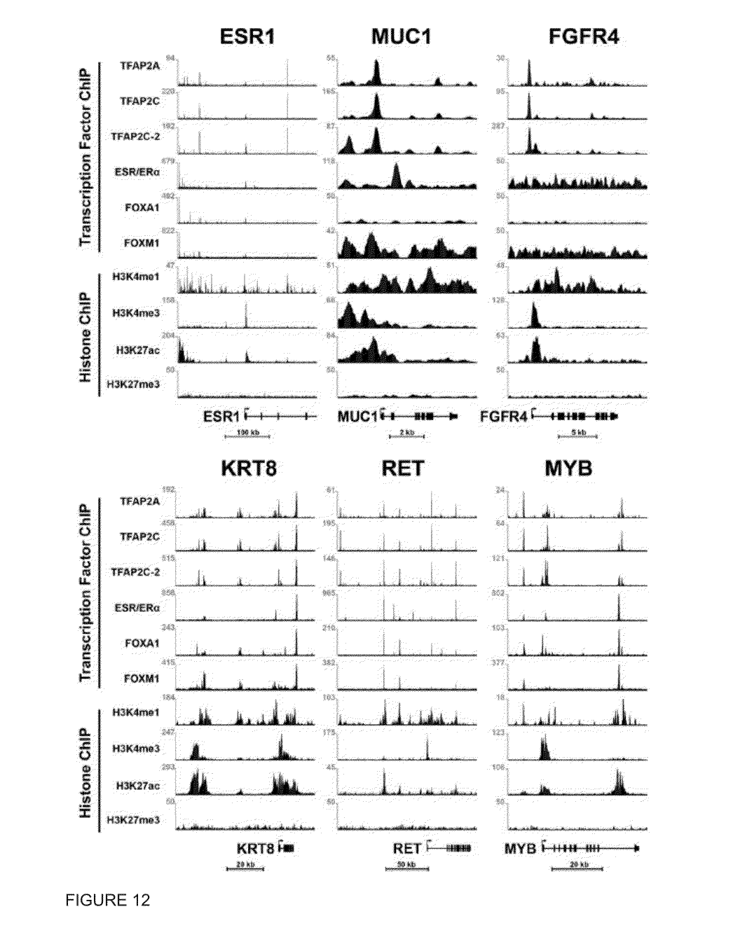

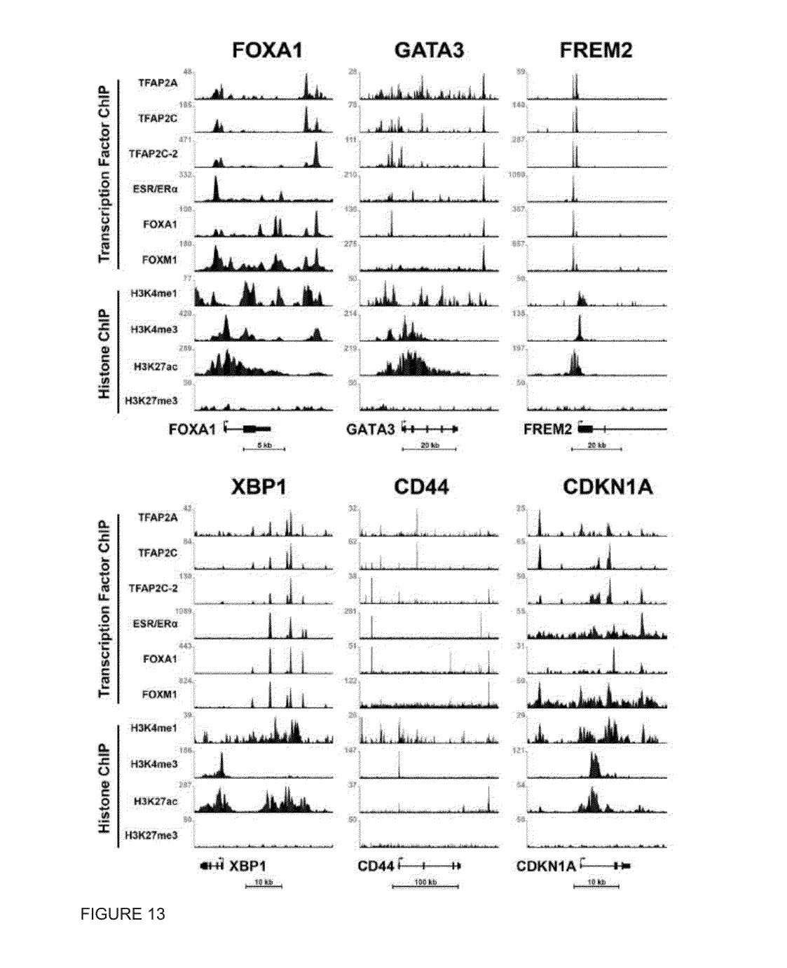

In addition to the genes examined above, the FREM2 gene was identified as a specific TFAP2C target gene, which was highly responsive to changes in TFAP2C expression but was unresponsive to TFAP2A (FIGS. 10 and 11). Using ChIP-seq the binding of TFAP2A and TFAP2C was compared in MCF-7 cells (FIG. 2A). An analysis of the genomic binding of TFAP2A and TFAP2C to the regulatory regions of the luminal associated genes, ESR1/ERα, FOXA1 and FREM2, demonstrated co-localization of the two factors. ChIP-seq data for the other luminal target genes examined demonstrated identical binding patterns for TFAP2A and TFAP2C in the promoter regulatory regions and these data agreed with other published ChIPseq data for MCF-7 cells (FIGS. 12 and 13). Epitope-tagged AP-2 constructs were used to confirm the identical chromatin binding patterns (FIG. 2B-C). HA tagged constructs for TFAP2A and TFAP2C were transfected into MCF-7 cells and ChIP was performed with anti-HA antibody with amplification at on-target and off-target sites for ESR1/ERα, FOXA1 and FREM2 (Woodfield et al., 2010). The data demonstrate that TFAP2A and TFAP2C bind to the AP-2 sites in the luminal target genes with approximately equal binding affinity. Hence, the functional differences between TFAP2A and TFAP2C cannot be attributed to differences in genomic binding.

Functional Specificity of TFAP2C Localized to Amino Terminus We sought to identify the domain of TFAP2C responsible for regulation of the luminal cluster genes. A knock-down/knock-in system was developed in which the expression of endogenous TFAP2C was knocked-down by siRNA and rescued by co-transfection with expression vectors for either TFAP2A or TFAP2C, engineered to be resistant to the siRNA. Chimeric AP-2 proteins were created where regions of TFAP2A were substituted with the homologous region of TFAP2C (FIG. 2D). Using endogenous ERα expression as a marker for activation, rescue of ERα expression was localized to the amino-half of TFAP2C (FIG. 2E). FREM2 was more robust as a marker for TFAP2C-specific activation and allowed localization of luminal-specific activation to the first 128 amino acids of the activation domain (FIG. 2F).

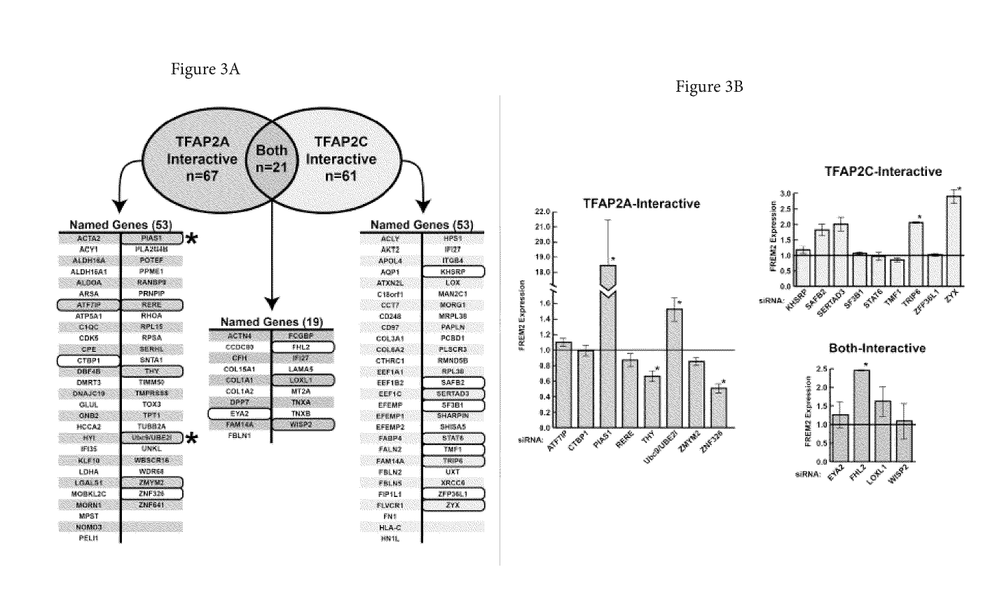

Sumoylation Pathway Maintains Specificity of TFAP2C. The luminal cluster gene promoters appear to share a common transcriptional mechanism and functional specificity of TFAP2C may involve either specific co-activator(s) for TFAP2C or promoter-specific co-repressors of TFAP2A. A set of potential AP-2 co-factors were identified using yeast two-hybrid in which either TFAP2C or TFAP2A was used as bait (FIG. 3A). A set of factors was chosen based on previous findings suggesting a potential role in gene regulation. The functional effect of each co-factor was assessed by serial knockdown using specific siRNAs and assaying for expression of endogenous FREM2 (FIG. 3B). Of 21 AP-2 binding partners tested, knockdown of two TFAP2A-interactive factors, PIAS1 and Ubc9/UBE2I significantly induced endogenous FREM2 expression in MCF-7 cells.

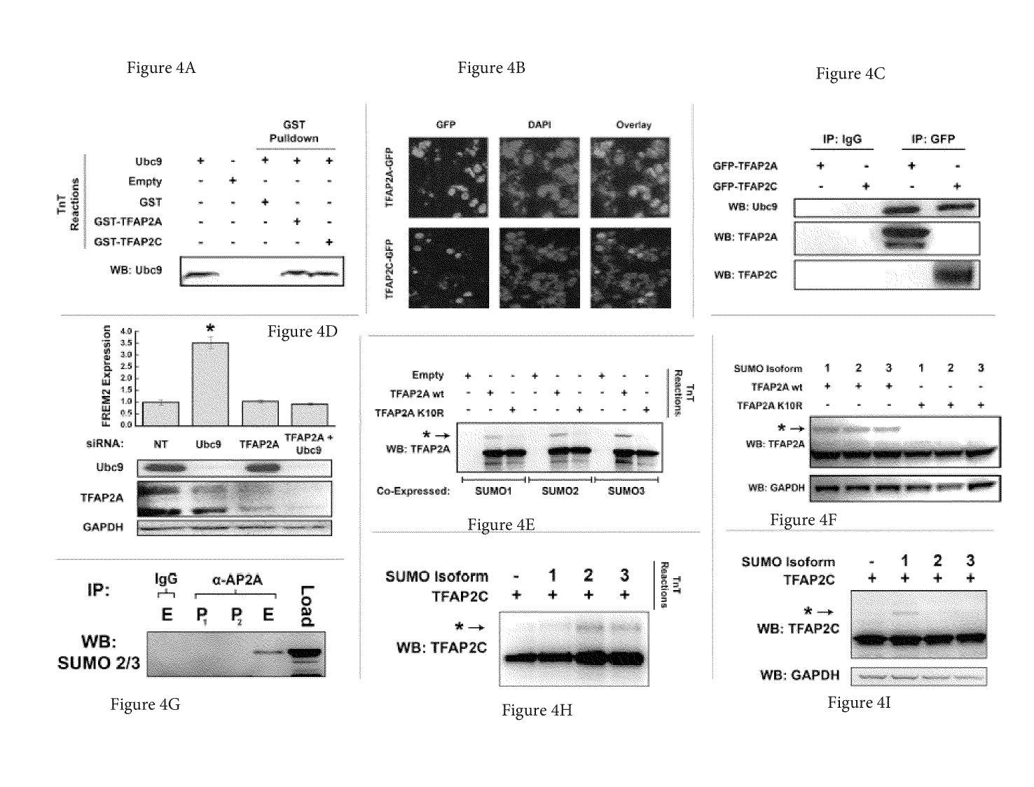

Ubc9 is a unique E2 SUMO conjugating enzyme (Ihara et al., 2008; Johnson and Blobel, 1997) and PIAS1 is a SUMO E3 ligase (Leitao et al., 2011). Ubc9 was previously shown to bind to TFAP2A and TFAP2C (Eloranta and Hurst, 2002). As both proteins are part of the sumoylation pathway, the findings implicated sumoylation as a mechanism accounting for specificity of TFAP2C in regulation of the luminal gene cluster. GST pull-down and co-immunoprecipitation confirmed that Ubc9 bound to both TFAP2A and TFAP2C (FIG. 4 A-C). Knockdown of Ubc9 increased endogenous FREM2 expression (FIG. 4D).

Whereas, knockdown of TFAP2A alone had no effect, knockdown of TFAP2A abrogated the effect of Ubc9 knockdown, indicating that in the absence of the sumoylation pathway, FREM2 expression became responsive to TFAP2A. Interestingly, Western blot of TFAP2A often demonstrates a doublet (FIGS. 11C, 4D and previous publications). Knockdown of Ubc9 reduced the relative amount of the upper band (FIG. 4D), suggesting sumoylation of TFAP2A. One major site for sumoylation is lysine 10 and the TFAP2A isoform 1A, which was used to generate the chimeric AP-2 proteins, contains the K10 SUMO site.

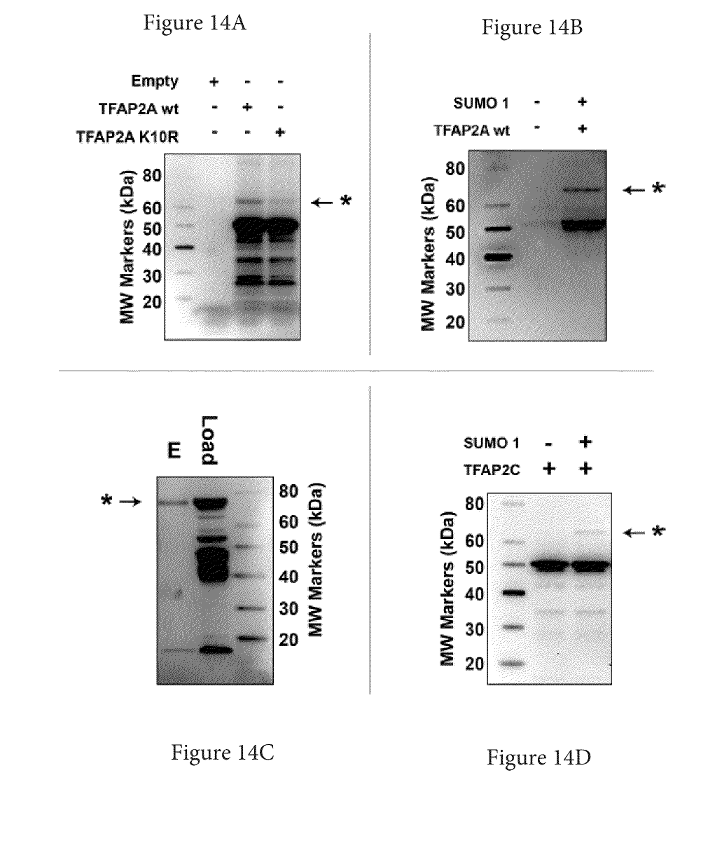

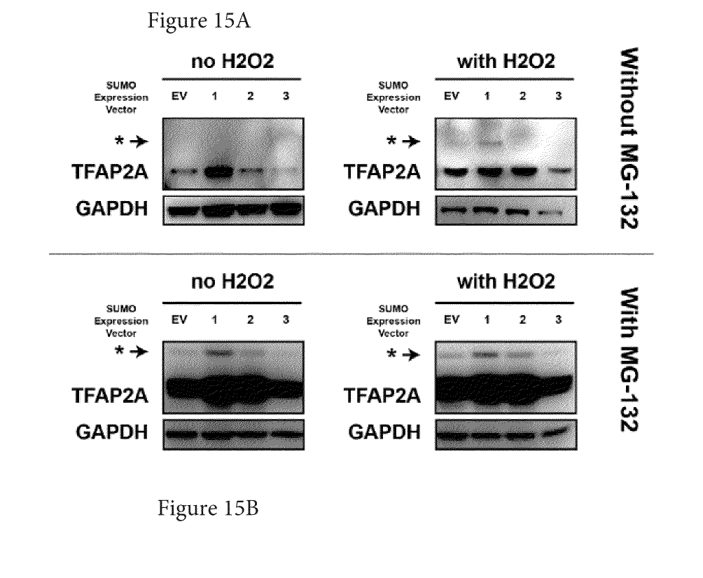

To demonstrate sumoylation of TFAP2A, SUMO-1, -2 and -3 were expressed in vitro with TFAP2A. Wild-type TFAP2A was sumoylated by all three SUMO proteins but the TFAP2A mutant K10R had significantly reduced sumoylation (FIG. 4E). In MCF-7 cells, wildtype TFAP2A but not K10R mutant was sumoylated in vivo with all three SUMO proteins (FIG. 4F). Immunoprecipitated TFAP2A was evaluated by western blot with anti-SUMO2/3 antibody and demonstrated the endogenous sumoylated form of TFAP2A in MCF-7 cells (FIG. 4G). Although sumoylated forms of TFAP2C were identified in vitro with SUMO-1, -2 or -3 (FIG. 4H), expression in MCF-7 cells in vivo was only able to identify sumoylation of TFAP2C with co-expression of SUMO-1 (FIG. 4I). The sumoylated forms of AP-2 were estimated to be 70±5 kDa (FIG. 14). Sumoylation of TFAP2A was similarly demonstrated in the basal line BT549, which was increased with peroxide (FIG. 15).

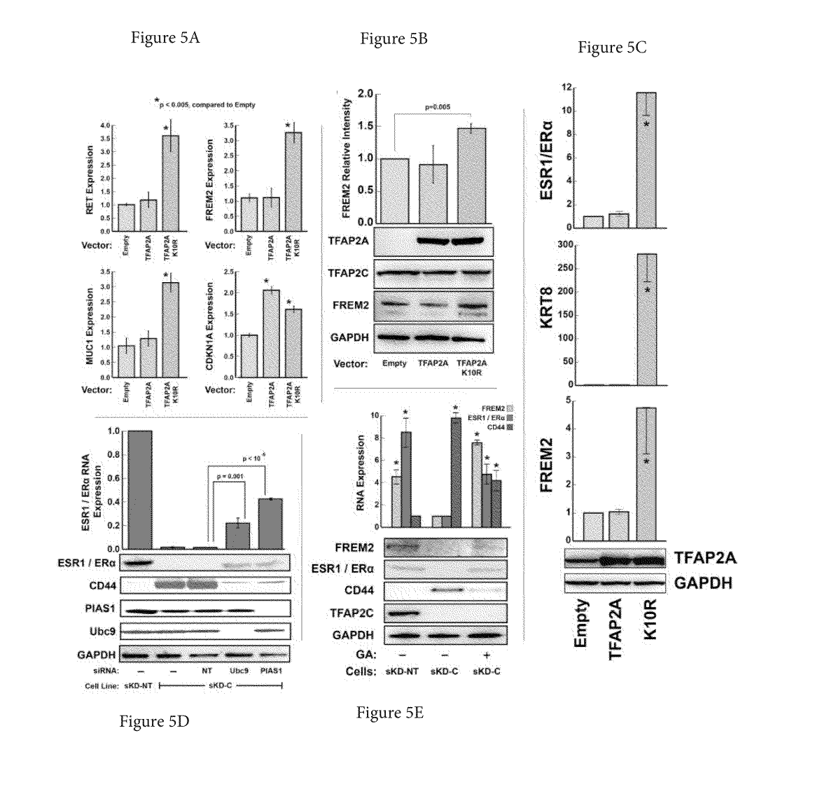

To demonstrate the functional effects of sumoylation, wild-type and the K10R mutant of TFAP2A were transfected into MCF-7 cells. Transfection of TFAP2A-K10R induced expression of luminal-associated genes (RET, MUC1 and FREM2), whereas, transfection of wild-type TFAP2A had no effect (FIG. 5A). By contrast, transfection of wild-type TFAP2A or K10R mutant induced expression of CDKN1A/p21-CIP. The induction of FREM2 protein with expression of TFAP2A-K1 OR was confirmed by Western blot, without changes in expression of TFAP2C (FIG. 5B). Though the effects on luminal gene expression were reproducible, the overall effects were modest since parental MCF-7 cells express TFAP2C as well as the luminal gene targets.

We have shown that stable knockdown of TFAP2C converts MCF-7 cells from a luminal to basal-like phenotype. Hence, a MCF-7 cell clone with stable knockdown of TFAP2C (sKD-C) was utilized compared to a cell clone with a non-targeting shRNA (sKD-NT). Overexpression of TFAP2A-K10R in sKD-C cells significantly induced ESR1/ERα, KRT8 and FREM2, whereas wild-type TFAP2A had no effect (FIG. 5C). Knockdown of either PIAS1 or Ubc9 in sKD-C cells resulted in re-activation of ER< mRNA and protein expression and repressed expression of the basal-associated gene CD44 (FIG. 5D). To confirm the effect of the sumoylation pathway, the small molecule inhibitor of sumoylation, ginkgolic acid (GA) (Fukuda et al., 2009), was examined for its effect on ERα, FREM2 and CD44 expression. Treatment of sKD-C cells with GA induced ERα and FREM2 expression and repressed CD44 expression compared to vehicle treatment without altering TFAP2C expression (FIG. 5E).

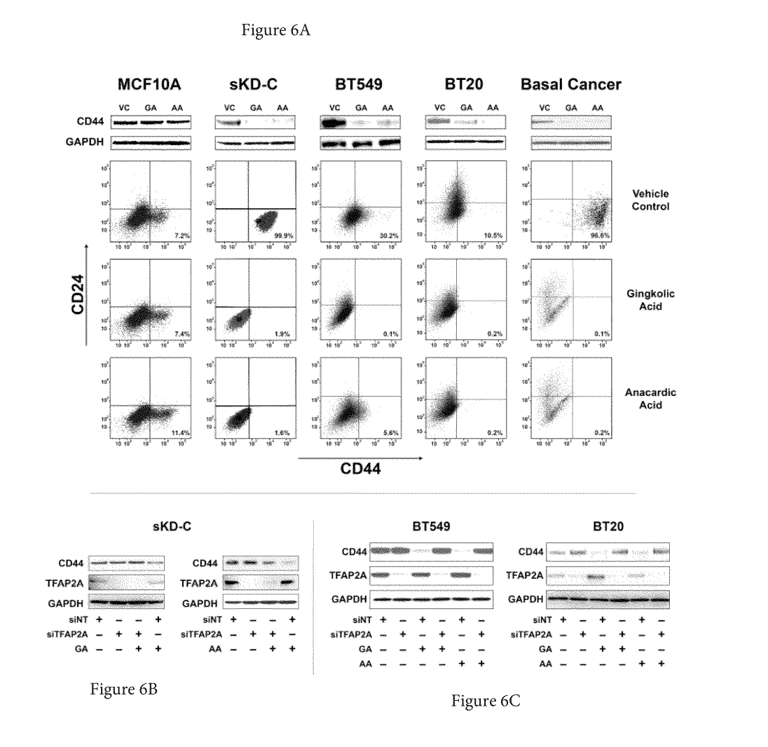

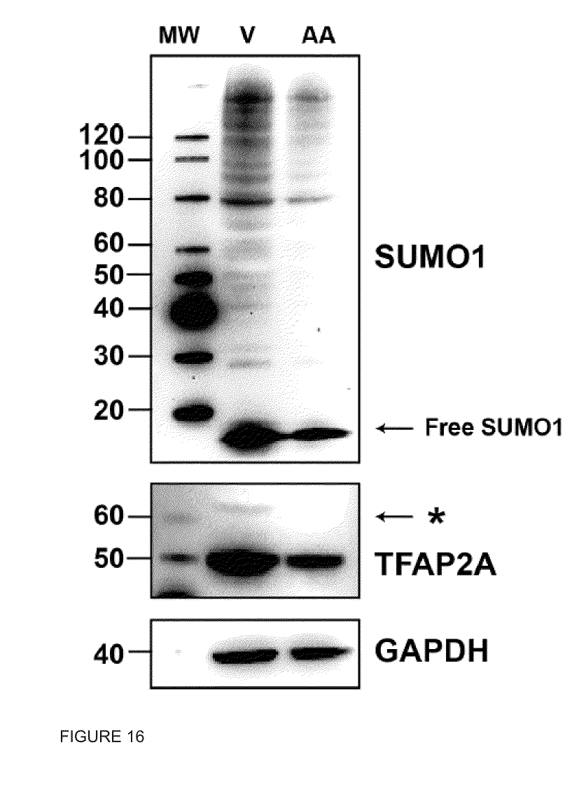

Sumoylation Inhibitors Clear the Cancer Stem Cell Population The findings suggest that the sumoylation pathway plays a critical role in maintaining the basal breast cancer phenotype. One of the characteristics of basal breast cancers is the relatively high percentage of cancer stem cells (CSC), identified using the characteristic markers of CD44^{+/hi}/CD24^{-/low}. To confirm the generality of the findings, basal breast cancer cell lines were treated with ginkgolic acid or another inhibitor of sumoylation, anacardic acid (AA) (Fukuda et al., 2009). AA inhibited SUMO conjugation of proteins globally and the SUMO conjugated form of TFAP2A specifically (FIG. 16). AA was also noted to decrease slightly the overall expression of TFAP2A. As seen in FIG. 1, knockdown of TFAP2C induced a 1.5-fold increase in TFAP2A, indicating that TFAP2C moderately represses TFAP2A. The finding that AA induced in a slight decrease in TFAP2A is consistent with the SUMO un-conjugated form of TFAP2A acquiring TFAP2C-like repression activity. Treatment of the basal breast cancer cell lines BT-20 and BT-549 as well as sKD-C cells with sumoylation inhibitors abrogated expression of CD44 and significantly reduced the CD44^{+/h}/CD24^{-/low} CSC population (FIG. 6A).

In addition, cells from a primary basal breast cancer were treated in vitro in parallel. The cancer was from a patient with locally advanced breast cancer that was refractory to conventional chemotherapy. Remarkably, treatment with GA or AA cleared the CD44^{+/hi}/CD24^{-/low} CSC population from the cells harvested from the primary tumor (FIG. 6A, last panel). By contrast, GA/AA treatment of MCF-10A cells, a normal breast cell line model, had no effect on CD44 expression and had no effect on the percentage of cells expressing stem cell markers.

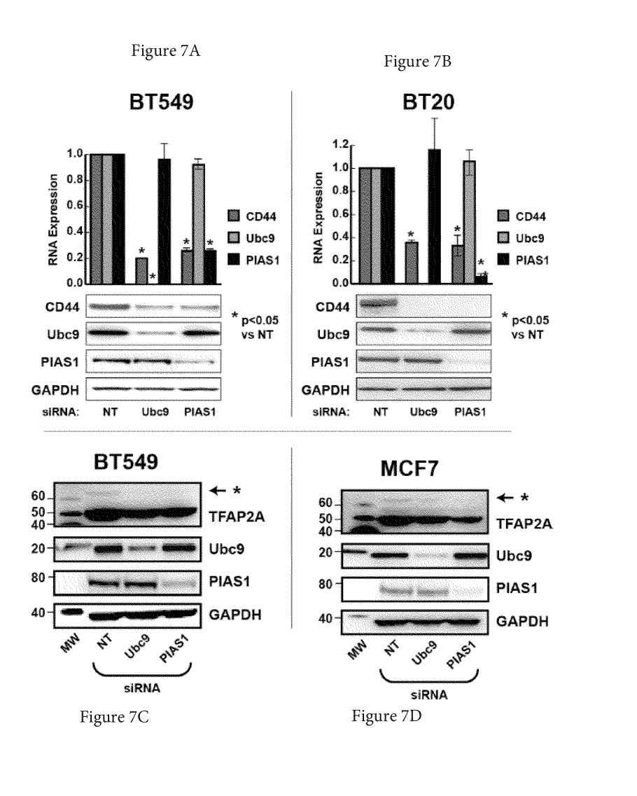

To further demonstrate the role of AP-2 in repression of CD44, the effects of GA and AA were examined with knockdown of TFAP2A. As seen in FIGS. 6B and 6C, knockdown of TFAP2A alone had no effect on CD44 expression in sKD-C, BT549 and BT20 cells. However, the ability for GA and AA to repress CD44 expression in the basal breast cancer cell lines was completely eliminated by knockdown of TFAP2A. Since drug effects might not be specific for a single pathway, GA and AA might induce changes in CD44 expression through several mechanisms. To prove that the effects on CD44 expression were mediated through the sumoylation pathway, Ubc9 and PIAS1 were knocked down by siRNA in the basal cell lines BT549 and BT20 and the effects on CD44 expression were assessed. As seen in FIGS. 7A and 7B, knockdown of either Ubc9 or PIAS1 similarly repressed CD44 expression. Furthermore, knockdown of Ubc9 or PIAS1 eliminated the SUMO conjugated form of TFAP2A (and slightly reduced the overall level of TFAP2A) in both basal (BT549) and luminal (MCF-7) cells (FIGS. 7C and 7D). The findings imply that SUMO inhibition cleared the CSC population.

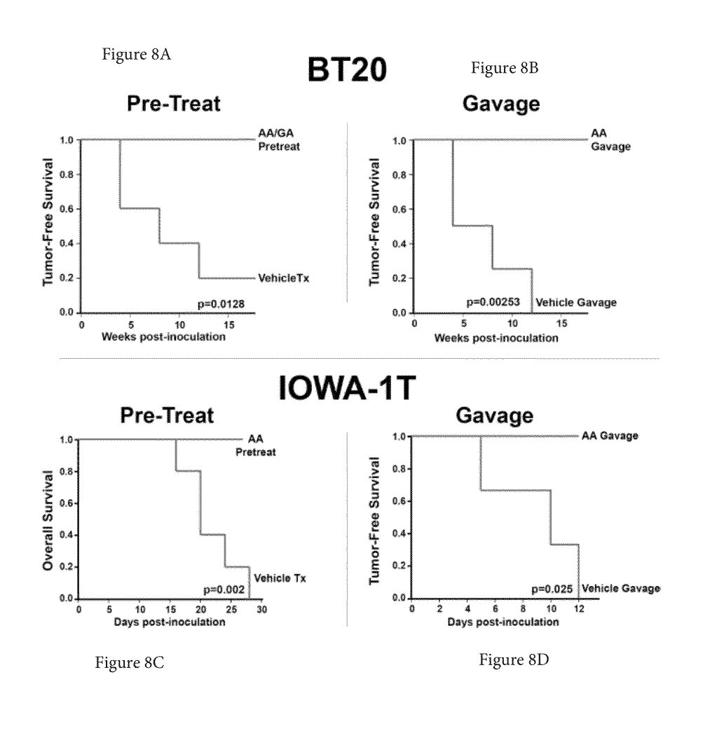

To address the effects of GA and AA on tumor initiation, BT20 cells were pretreated with GA or AA compared to vehicle prior to inoculation of xenografts in nude mice. As noted in FIG. 8A, pretreatment of the cells repressed the formation of tumor xenografts, whereas, vehicle treated cells formed xenografts with a median time of 8 weeks. To prove that the drugs were not cytotoxic, BT20 xenografts were inoculated in nude mice and the animals were gavaged with AA or vehicle. As noted in FIG. 8B, animals gavaged with AA failed to form tumors, whereas, vehicle gavaged animals formed xenografts as expected. We created a breast carcinoma cell line (IOWA-1T) from the basal tumor-derived cells used in FIG. 6A. The IOWA-1T cell line rapidly forms locally advanced tumors in nude mice. Identical experiments using the IOWA-1T basal cell line confirmed that either pre-treating the cancer cells or gavaging animals with AA repressed tumor initiation of xenografts (FIGS. 8C and 8D).

### Discussion

Transcriptional Mechanisms Regulating the Luminal Phenotype The current study establishes that TFAP2C has a distinct functional role in regulation of the luminal gene expression cluster. Further, the cell line models are reflective of gene regulation in primary ERα-positive cancer. Several lines of evidence indicate that sumoylation plays a key role in establishing the functional differences between TFAP2C and TFAP2A. First, we have demonstrated sumoylation of TFAP2A at lysine 10 in vitro and in vivo. Second, blocking the sumoylation pathway either by knockdown of critical enzymes in the sumoylation pathway or with the use of small molecule inhibitors of sumoylation allowed TFAP2A to induce expression of luminal genes such as ESR1/ERα and FREM2 and repress expression of the basal gene CD44. Finally, mutation of the SUMO target lysine of TFAP2A conferred the ability to induce expression of luminal cluster genes.

Taken together the data indicate that sumoylation of endogenous TFAP2A blocks this factor from regulating luminal gene expression and forms the basis for the unique role of TFAP2C in luminal gene regulation. Furthermore, it is clear that the functional effect of sumoylation is a luminal gene-specific effect since wild-type or K1 OR mutant TFAP2A was active in transcriptional activation of CDKN1A/p21-CIP. Although there is evidence that TFAP2C can be sumoylated, we were not able to demonstrate that sumoylation has functional effects on the transcriptional activity of TFAP2C. Under conditions where sumoylation of TFAP2A blocked its activity at luminal gene promoters, TFAP2C remained active despite evidence for similar levels of sumoylation. TFAP2C participates in luminal mammary development and luminal gene expression in breast cancer, further strengthening the link between the processes of luminal differentiation and oncogenesis.

The Role of Sumoylation in Cancer Related Gene Regulation. Sumoylation involves the post-translational modification of proteins through the covalent attachment of small ubiquitin-like modifiers (SUMO) proteins to lysine residues in target proteins. At least four SUMO proteins have been described, SUMO-1-4. The enzymatic pathway involves several steps beginning with ATP-dependent activation of the SUMO protein by the E1 heterodimer ASO1-UBA2, continuing with the transfer of SUMO to the cysteine residue of the E2 enzyme Ubc9, and finally the enzymatic transfer of the SUMO tag to the target protein by the E3 ligase, e.g. PIAS1. Sumoylation of key regulatory proteins influences several aspects of oncogenesis and cancer progression. There is a growing body of literature reporting the sumoylation of transcription factors and the effects on transcriptional regulation (Gill, 2003) including sumoylation of androgen receptor (AR), glucocorticoid receptor (GR), C/EBP, Smad4, Myb, Ets-1, Pdx1, Sp3, p300, CREB and p53 (Bettermann et al., 2012). In most cases, sumoylation represses transcriptional activity. Mechanisms resulting in transcriptional repression by sumoylation may include effects of protein stability, altered cellular localization or DNA binding, modulation of co-repressor binding and altered association with chromatin modifying enzymes such as histone deacetylases (HDACs) (Gill, 2003; Girdwood et al., 2003). Holmstrom et al. (Holmstrom et al., 2008) showed that transcriptional inhibition by sumoylation occurred at compound, but not single, sites was related to the ability for sumoylation to destabilize the transcription factor-chromatin interaction.

A mechanism whereby sumoylation destabilizes TFAP2A binding to certain regulatory regions may provide a mechanism for promoter-specific repression. Since the function of TFAP2A at promoters for genes such as CDKN1A/p21-CIP is SUMO-insensitive, promoter regulatory structure common to luminal genes may account for SUMO-specific effects. Many luminal genes contain closely linked promoter elements for AP-2, ERα and FOXA1 (Tan et al., 2011), and the interaction of these factors may be sensitive to sumoylation. Consistent with previous studies, sumoylation of TFAP2A was increased by peroxide and confirms that oxidative stress can increase sumoylation of factors (Bossis and Melchior, 2006; Ryu et al., 2010). Recent studies have suggested that the sentrin-specific protease 1 (SENP1) may be involved in SUMO de-conjugation of factors in breast cancer (Abdel-Hafiz and Horwitz, 2012; Chen et al., 2013).

Sumoylation Inhibitors Clear the Cancer Stem Cell Compartment. Basal breast cancers are characterized by a relatively high percentage of CSCs identified as the cell population expressing CD44⁺/CD24^{-/low} (Al-Hajj et al., 2003; Iqbal et al., 2013). The CSC population is relatively chemo-resistant and becomes enriched after chemotherapy (Lee et al., 2011). Stable knockdown of TFAP2C in MCF-7 cells repressed luminal gene expression and increased the population of cells expressing CSC markers.

In the current study, SUMO inhibition allowed TFAP2A to acquire TFAP2C-like repression activity, inhibiting CD44 expression, clearing cells expressing CSC markers and blocking the outgrowth of cancer xenografts. Of particular clinical relevance, sumoylation inhibitors were able to efficiently clear the CSC population in a primary basal breast cancer obtained from a patient with a locally advanced breast cancer that was refractory to conventional chemotherapy. The high percentage of cells with CSC markers was likely due to selection from the treatment with chemotherapy. The remarkable effect of SUMO inhibitors to clear the CSC population demonstrates that this class of agents can be developed as novel cancer drugs either alone or in combination with conventional chemotherapy. Since the CD44^{+/hi}/CD24^{-/low} population defines CSCs in many types of carcinomas, SUMO inhibitors can have clinical effects in a wide range of carcinomas. Interestingly, sumoylation inhibitors did not affect MCF10A cells, which are commonly used as a model for normal breast cells. Hence, the sumoylation pathway are critical for maintaining the basal breast cancer subtype, and is not a general mechanism regulating CD44 expression in normal breast cells.

### Experimental Procedures

Cell Lines

The human breast cancer cell lines (MCF-7, SKBR-3, BT-20, BT-549, MCF-10A) were obtained from American Type Culture Collection. sKD-C cells were subcloned from parental MCF-7 cells with stable knockdown of TFAP2C as described (Cyr et al., 2014). ER<-positive tumors and the basal cancer tumor were obtained from surgical resection specimens. Cell suspensions were prepared with gentle collagenase/hyaluronidase (Stemcells) (Ponti et al., 2005). A new cell line was established from the basal tumor cells, called IOWA-1T (manuscript in preparation).

RNA Isolation, cDNA Synthesis and Real-Time-PCR

Total RNA was isolated and analyzed as previously described. Expression values were normalized to the mean of 18S rRNA 4319413E-0803037 (Applied Biosystems) as endogenous control.

Chromatin Immunoprecipitation with Direct Sequencing (ChIP-Seq)

ChIP-Seq was performed as described (Woodfield et al., 2010). The ChIP-Seq data are accessible in GEO database under accession number GSE44257. Details of real-time ChIP have been described (Woodfield et al., 2010).

Transient Transfection

Transfections were performed as previously described (Woodfield et al., 2007) and siRNAs for non-targeting (Dharmacon), Ubc9/UBE2I siRNA (Ambion) and PIAS1 (Ambion) were used.

Sumoylation Assays

Sumoylation in vitro was done using SUMOlink™ SUMO-1 Kit and SUMOlink™ SUMO-2/3 Kit (Carlsbad). SUMO plasmids and pcDNA3.1-TFAP2A or K10R mutant were used for in vitro protein production. MCF-7 were transfected for 48 hours with 2 <g SUMO expressing plasmids (Feng et al., 2013) that were kindly provided by Dr Xiaolu Yang (University of Pennsylvania). As indicated in some experiments, the proteasome inhibitor MG132 was added as described (Chu and Yang, 2011).

Western Blots

Western blots were performed as previously described (Cyr et al., 2014; Kulak et al., 2013). For immunoprecipitations anti-TFAP2A, anti-GFP antibody (Life Technologies) or IgG (#sc-2025, Santa Cruz Biotechnology) were cross-linked with Dynabeads protein A (Life Technologies). MCF7 protein was incubated with Dynabeads protein A-Abs complex, protein complexes were washed with DPBS (Gibco) with 0.01% Tween (Research Products International Corp.), and eluted with SDS loading buffer.

AP-2 Constructs

AP-2 constructs were amplified using previously cloned cDNAs for template (McPherson and Weigel, 1999). HA-tagged TFAP2C was prepared using a primer pair; HA tagged TFAP2A using a primer pair; both cDNAs were cloned into the pcDNA3.1 Nhe1/Xba1 sites. For GFP-tagged AP-2 constructs, primers for TFAP2A and TFAP2C were cloned into pCRγ8/GW/TOPO® TA (Invitrogen) using TOPO cloning kit (Invitrogen). Gateway TFAP2A and TFAP2C clones were inserted in-frame into pGLAP1 (Torres et al., 2009) via LR clonase reaction using Gateway LR Clonase II Enzyme Mix (Invitrogen). To generate CAD-ADBD, primers were used to amplify TFAP2C activation domain from TFAP2C pcDNA3.1+ construct. After BamHI and EarI digestion, the PCR product was ligated into pcDNA3.1+(Invitrogen). Primers were used to amplify TFAP2A DNA binding domain. After XhoI and EarI digestion, the PCR product was ligated into CAD-pcDNA3.1+ plasmid. To generate AAD-CDBD, primers were used to amplify TFAP2A activation domain from TFAP2A pcDNA3.1+. After KpnI and EarI digestion, the PCR product was ligated into pcDNA3.1+. Primers were used to amplify TFAP2C DNA binding domain digested with EarI and XhoI and ligated into AAD−pcDNA3.1+ plasmid. The primers amplified the aad region, and cdbd. After digestion with KpnI, EarI and XhoI PCR products were ligated into pcDNA3.1+ within KpnI and XhoI sites. To amplify cad-adbd, primers amplified the N-terminal part of TFAP2C, and TFAP2A DNA binding domain. After digestion with BamHI, EarI and XhoI enzymes PCR products were ligated into pcDNA3.1+. To obtain K10R-pcDNA3.1+ plasmid primers were used to amplify the coding region of TFAP2A-pcDNA3.1+. After the initial PCR reaction the template DNA was digested away with DpnI.

TnT Reaction and GST Pull Down

The coding region of Ubc9/UBE2I was amplified from pACT2-Ubc9/UBE2I using primers and ligated into pcDNA3.1+(Invitrogen). GST pull down was done using ProFound Pull-Down GST kit (ThermoScientific).

Gingolic and Anacardic Acid Treatment.

Cells were plated (2.5×10₅/10 cm₂) and treated with 10 μM gingolic or anacardic acid (Sigma) for 2-4 days and collected for qPCR, Western blot and FACS analysis.

Flow Cytometry

FACS analysis was performed as previously described (Roederer and Hardy, 2001).

Yeast Two-Hybrid Assay

Yeast two-hybrid for AP-2 factors was performed as previously described (McPherson et al., 2002). Inserts in the yeast plasmids were PCR amplified with the vector-specific primers.

Tumor Xenografts

Xenografts were generated by inoculating 5×10⁶ BT20 cells into nude mice as previously described (Woodfield et al., 2007).

Statistical Analysis

Statistical analysis was performed using the two-sided Student's T-test for continuous variables. Comparisons for xenografts were performed using the log rank test.

### Example 2

### Stem Cells Marker Downregulation in Colorectal, Pancreatic, Thyroid Carcinomas after SUMO Inhibitor Treatment

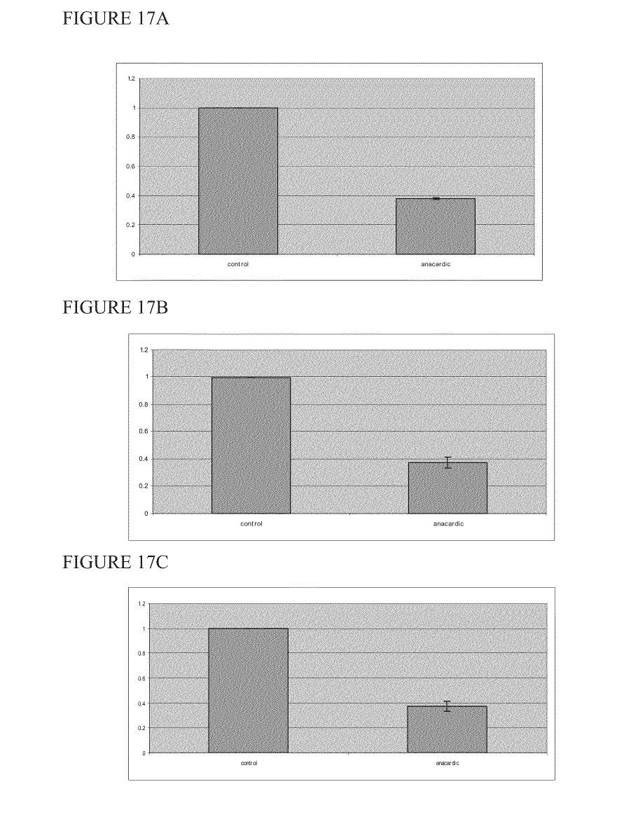

Informed consent primary colorectal tumor material was obtained during operation and was transferred to lab. Colorectal cancer stem cells (CSC) were isolated accordingly Cammareri et al, 2008 and treated with 10 μM anacardic acid within 48 hours. It was shown by qPCR that colorectal stem cells markers CD44, ALCAM and EPCAM were down regulated (FIG. 17A-C), CD44 reduction was proved by Western blot (FIG. 17D).

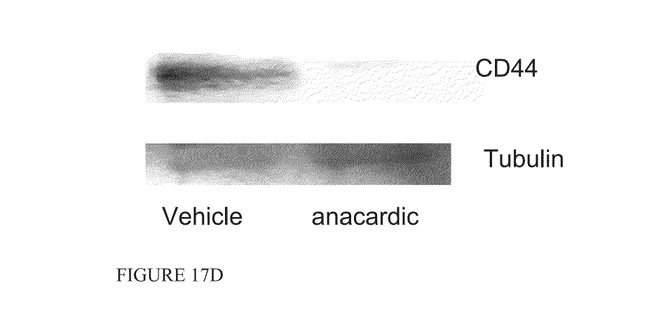

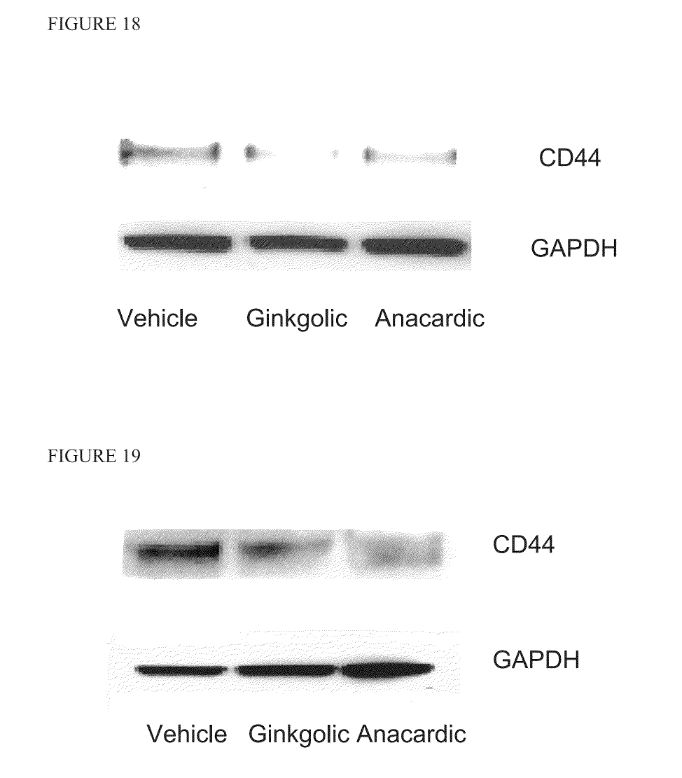

Similar downregulation of stem cell marker CD44 was determined in pancreatic carcinoma Panc-1 cell line (FIG. 18) and undifferentiated thyroid cell line 8505c (FIG. 19) after 96 h treatment with SUMO inhibitors anacardic and ginkgolic acids.
Woodfield, G. W., Horan, A. D., Chen, Y. & Weigel, R. J. TFAP2C controls hormone response in breast cancer cells through multiple pathways of estrogen signaling. Cancer Res 67, 8439-8443 (2007).
Woodfield, G. W., Chen, Y., Bair, T. B., Domann, F. E. & Weigel, R. J. Identification of primary gene targets of TFAP2C in hormone responsive breast carcinoma cells. Genes Chromosomes Cancer 49, 948-962, doi:10.1002/gcc.20807 (2010).
Tan, S. K. et al. AP-2gamma regulates oestrogen receptor-mediated longrange chromatin interaction and gene transcription. Embo J 30, 2569-2581, doi:10.1038/emboj.2011.151 (2011).
Sorlie, T. et al. Gene expression patterns of breast carcinomas distinguish tumor subclasses with clinical implications. Proc Natl Acad Sci USA 98, 10869-10874. (2001).
Kao, J. et al. Molecular profiling of breast cancer cell lines defines relevant tumor models and provides a resource for cancer gene discovery. PLoS One 4, e6146, doi:10.1371/journal.pone.0006146 (2009).
McPherson, L. A., Baichwal, V. R. & Weigel, R. J. Identification of ERF-1 as a member of the AP2 transcription factor family. Proceedings of the National Academy of Sciences of the United States of America 94, 4342-4347 (1997).
Hoffman, T. L., Javier, A. L., Campeau, S. A., Knight, R. D. & Schilling, T. F. Tfap2 transcription factors in zebrafish neural crest development and ectodermal evolution. J Exp Zool B Mol Dev Evol 308, 679-691, doi:10.1002/jez.b.21189 (2007).
Scibetta, A. G., Wong, P. P., Chan, K. V., Canosa, M. & Hurst, H. C. Dual association by TFAP2A during activation of the p21cip/CDKN1A promoter. Cell Cycle 9, 4525-4532 (2010).
Ihara, M., Stein, P. & Schultz, R. M. UBE2I (UBC9), a SUMO-conjugating enzyme, localizes to nuclear speckles and stimulates transcription in mouse oocytes. Biol Reprod 79, 906-913, doi:10.1095/biolreprod.108.070474 (2008).
Leitao, B. B., Jones, M. C. & Brosens, J. J. The SUMO E3-ligase PIAS1 couples reactive oxygen species-dependent JNK activation to oxidative cell death. Faseb J, doi:10.1096/fj.11-186346 (2011).
Eloranta, J. J. & Hurst, H. C. Transcription factor AP-2 interacts with the SUMO-conjugating enzyme UBC9 and is sumolated in vivo. The Journal of biological chemistry 277, 30798-30804, doi:10.1074/jbc.M202780200 (2002).
Berlato, C. et al. Alternative TFAP2A isoforms have distinct activities in breast cancer. Breast Cancer Res 13, R23, doi:10.1186/bcr2838 (2011).
Fukuda, I. et al. Ginkgolic acid inhibits protein SUMOylation by blocking formation of the E1-SUMO intermediate. Chem Biol 16, 133-140, doi:10.1016/j.chembiol.2009.01.009 (2009).
Lee, H. E. et al. An increase in cancer stem cell population after primary systemic therapy is a poor prognostic factor in breast cancer. Br J Cancer 104, 1730-1738, doi:10.1038/bjc.2011.159 (2011).
Bettermann, K., Benesch, M., Weis, S. & Haybaeck, J. SUMOylation in carcinogenesis. Cancer Lett 316, 113-125, doi:10.1016/j.canlet.2011.10.036 (2012).
Gill, G. Post-translational modification by the small ubiquitin-related modifier SUMO has big effects on transcription factor activity. Curr Opin Genet Dev 13, 108-113 (2003).
Girdwood, D. et al. P300 transcriptional repression is mediated by SUMO modification. Mol Cell 11, 1043-1054 (2003).
Holmstrom, S. R., Chupreta, S., So, A. Y. & Iniguez-Lluhi, J. A. SUMOmediated inhibition of glucocorticoid receptor synergistic activity depends on stable assembly at the promoter but not on DAXX. Molecular endocrinology 22, 2061-2075, doi:10.1210/me.2007-0581 (2008).
Feng, Z. J., Gurung, B., Jin, G. H., Yang, X. L. & Hua, X. X. SUMO modification of menin. American journal of cancer research 3, 96-106 (2013).
McPherson, L. A., Loktev, A. V. & Weigel, R. J. Tumor Suppressor Activity of AP2alpha Mediated through a Direct Interaction with p53. J Biol Chem 277, 45028-45033. (2002).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for treatment of cancer, the method comprising:
contacting a cancer cell population with a sumoylation inhibitor in a dose effective to reduce the number of cancer cells in the population.

2. The method of claim 1, wherein the dose of sumoylation inhibitor is effective to reduce levels of sumo-conjugated TFAP2A.

3. The method of claim 2, wherein the method reduces the number of cancer stem cells, and reduction of cancer stem cells is monitored by determining the expression of CD44+ cells in the population.

4. The method of claim 1, further comprising contacting the cancer cell population with one or more chemotherapeutic agents, wherein the population of cells has increased responsiveness to chemotherapeutic agents following contacting with a sumoylation inhibitor.

5. The method of claim 1, further comprising treating the cancer cell population with radiation therapy, wherein the population of cells has increased responsiveness to radiation therapy following contacting with a sumoylation inhibitor.

6. The method of claim 1, wherein the cancer cell population is a human cell population.

7. The method of claim 6, wherein said cancer is a solid tumor.

8. The method of claim 6, wherein said tumor is a carcinoma.

9. The method of claim 8, wherein said tumor is a breast carcinoma.

10. The method of claim 9, wherein the cancer cell population is a basal-type breast carcinoma, and wherein following contacting with the sumoylation inhibitor the basal-type carcinoma cells are induced to a luminal-type breast carcinoma phenotype.

11. The method of claim 10, further comprising the step of monitoring expression of at least one marker indicative of the breast carcinoma phenotype.

12. The method of any one of claims 1-11, wherein the sumoylation inhibitor is anacardic acid or a derivative or analog thereof.

13. The method of claim 12, wherein the sumoylation inhibitor is gingkolic acid.

14. A method of screening for a candidate agent that is a sumoylation inhibitor useful in treatment of cancer, the method comprising:
contacting a cancer cell with the candidate agent; and
determining the concentration of sumo-conjugated TFAP2A, wherein a reduction in sumo-conjugated TFAP2A is indicative that the candidate agent is useful in cancer treatment.

15. The method of claim 14, further comprising:
contacting a population of cancer cells with the candidate agent; and
determining the concentration of cancer stem cells, wherein a reduction in the number of cancer stem cells is indicative that the candidate agent is useful in cancer treatment.

16. The method of claim 15, wherein the cancer stem cells are monitored by expression of CD44.
